(19) United States
(12) Patent Application Publication
(10) Pub. No.: US 2010/0255054 A1
(43) Pub. Date: Oct. 7, 2010

(54) PHARMACEUTICAL COMPOSITION FOR TREATMENT AND PREVENTION OF RESTENOSIS

(76) Inventors: Taehwan Kwak, Gyeonggi-do (KR); Sang-ku Yoo, Gyeonggi-do (KR); Myung-gyu Park, Gyeonggi-do (KR)

Correspondence Address: BIRCH STEWART KOLASCH & BIRCH, PO BOX 747, FALLS CHURCH, VA, 22040-0747, US

(21) Appl. No.: 12/515,015

(22) PCT Filed: Nov. 26, 2007
(86) PCT No.: PCT/KR07/06011

§ 371 Date: May 15, 2009

(30) Foreign Application Priority Data
Nov. 27, 2006 (KR) 10-2006-0117685
Oct. 24, 2007 (KR) 10-2007-0107041

(51) Int. Cl. A61K 9/14; C07D 311/92; A61K 31/352; C07D 307/92; A61K 31/343; C07D 311/96; C07D 335/08; A61K 31/382; C07D 333/74; A61K 31/381; C07D 327/06; A61K 31/39; A61P 9/10

(52) U.S. Cl. 424/423; 549/389; 514/454; 424/489; 549/458; 514/468; 549/384; 514/453; 549/331; 549/26; 514/437; 549/43; 514/443; 549/16; 514/434; 549/457

(57) ABSTRACT

Provided is a pharmaceutical composition for the treatment and/or prevention of restenosis including (a) a therapeutically effective amount of a particular compound represented by Formula 1 and 2, or a pharmaceutically acceptable salt, prodrug, solvate or isomer thereof, and (b) a pharmaceutically acceptable carrier, a diluent or an excipient, or any combination thereof.

### FIELD OF THE INVENTION

The present invention relates to a pharmaceutical composition having therapeutic effect on the treatment and/or prevention of restenosis by inhibiting vascular smooth muscle cell proliferation, and more particularly to a pharmaceutical composition including (a) a therapeutically effective amount of a compound represented by Formula 1 and Formula 2, or a pharmaceutically acceptable salt, prodrug, solvate or isomer thereof, and (b) a pharmaceutically acceptable carrier, a diluent or an excipient, or any combination thereof.

### BACKGROUND OF THE INVENTION

Vascular smooth muscle cell proliferation, which is a response to vessel wall injury, refers to a phenomenon observed conspicuously in arteriosclerosis which shows secondary changes in the arterial intima due to vessel wall injury caused by lipids. Thus, vascular smooth muscle cell proliferation is known to be a leading cause of atherosclerosis. Moreover, vascular smooth muscle cell proliferation is known serious problem, considerably observed after a surgical operation, such as angioplasty, bypass surgery or vascular graft, which is the best method hi the present for recovering functions of blood vessels that have been narrowed by arteriosclerosis.

Therefore, in order to prevent and treat arteriosclerosis, vascular smooth muscle cell proliferation is considered to be a very important factor and as a result, research on the vascular smooth muscle cell proliferation is currently carried out actively. Recently, effects of AMP-activated protein kinase (AMPK), known to play a crucial role in cell energy metabolism, have been reported in that increased AMPK activity serves to inhibit vascular smooth muscle cell proliferation.

In this connection, NAD is an important factor among various factors for increasing AMPK activity, and NAD(P)H:quinine oxidoreductase 1 (NQO1) is one of the main factors for elevating NAD in cells.

NAD(P)H:quinone oxidoreductase (EC1.6.99.2) is also called as DT-diaphorase, quinone reductase, menadione reductase, vitamin K reductase, or azo-dye reductase, and such NQO is present in two isoforms, designated as NQO1 and NQO2 (ROM. J. INTERN. MED. 2000-2001, Vol. 38-39, 33-50). NQO is a flavoprotein and catalyzes two electron reduction and detoxification of quinone or quinone derivatives. The activity of NQO prevents formation of very highly-reactive quinone metabolites, detoxifies benzo(d)pyrenes and quinones, and diminishes the toxicity of chromium. The activity of NQO is found in all kinds of tissues, but varies from tissue to tissue. Generally, high-level expression of NQO was confirmed in cancer cell, liver, stomach and kidney tissues. NQO gene expression is triggered by xenobiotics, anti-oxidants, oxidants, heavy metals, UV light, radiation exposure, or the like. NQO is a part of numerous cellular defense mechanisms induced by oxidative stress. Associated expressions of genes implicated in such cellular defense mechanisms, including NQO gene expression, serve to protect cells against oxidative stress, free radicals and neoplasia.

NQO1 is largely distributed in epithelial and endothelial cells. This implies that NQO1 can act as a defense mechanism against compounds absorbed via air, the esophagus or blood vessels. Recent research shows participation of NQO1 in stabilization of the cell cycle regulating p53 through the redox mechanism.

NQO utilizes both of NADH and NADPH as an electron donor. While NADPH is used as a reducing agent in biosynthetic processes, NADH is used in energy-producing reactions. NADPH is an important factor implicated in fat synthesis, and the synthesis of palmitate requires 14 NADPH molecules.

However, free radicals such as reactive oxygen species (ROS) are generated, during which surplus NAD(P)H, remained after fat synthesis and energy production, is scavenged by an oxidative enzyme, called NAD(P)H oxidase, present on a plasma membrane. A primary cause of increased oxidative stress in obesity and diabetic diseases has been found to be NAD(P)H oxidase (Free Radical Biology & Medicine. Vol. 37, No 1, 115-123, 2004). It has been also found that free radicals such as reactive oxygen species (ROS) generated by NAD(P)H oxidase are main factors responsible for pathogenesis of various diseases such as cancers, cardiovascular diseases, hypertension, arteriosclerosis, cardiac hypertrophy, ischemic heart diseases, septicemia, inflammatory conditions and diseases, thrombosis, cranial nerve diseases (such as cerebral apoplexy (stroke), Alzheimer's disease, and Parkinson's disease), senescence-acceleration (J. Pharm. Phaimacol. 2005, 57 (1):111-116).

Therefore, when NAD⁺/NADH and NADP⁺/NADPH ratios in vivo or in vitro decrease, thereby remaining surplus NADH and NADPH molecules, they are utilized in a fat biosynthesis process. In addition, since surplus NADH and NADPH are also used as main substrates causing generation of reactive oxygen species (ROS) when they are present in excessive amounts, NADH and NADPH may be a pathogenic factor for significant diseases including inflammatory conditions and diseases caused by ROS. For these reasons, it is believed that fat oxidation and various energy expenditure (metabolism) by NAD⁺ and NADP⁺ will be activated when an in vivo or in vitro environment can be established to ensure stable maintenance of NAD⁺/NADH and NADP⁺/NADPH ratios in an increased state.

There has recently been a great deal of attention devoted to NA(D)P⁺. NA(D)P⁺ functioning as a substrate or a coenzyme for various enzymes involved in numerous metabolisms including fat oxidation. Specifically, NA(D)P⁺ is an in vivo substance implicated in numerous biological metabolic processes and is used as a substrate or a coenzyme for various kinds of enzymes including NAD⁺-dependent DNA ligase, NADtdependent oxidoreductase, poly(ADP-ribose) polymerase (PARP), CD38, AMPK, CtBP and Sir2p family members, as well as used as a coenzyme of various enzymes responsible for regulation of energy metabolism, DNA repair and transcription. NAD⁺ was found to play a crucial role in transcriptional regulation, longevity, calorie restriction-mediated diseases through the above-mentioned in vivo actions.

Therefore, the NAD(P)⁺/NAD(P)H ratio, a key factor regulating intracellular redox state, is often regarded as an indicator reflecting the metabolic state of organisms. The NAD(P)⁺/NAD(P)H ratio varies with changes in the metabolic process. Inter alia, it is known that NAD⁺ functions as a metabolic regulator. A variety of aging-related diseases are directly or indirectly associated with changes in the redox state of NAD⁺ or NAD(P)⁺/NAD(P)H.

Meanwhile, it was confirmed that AMP-activated protein kinase (AMPK) is a protein that senses the energy status, degree of redox state and phosphorylation in living organisms, and is activated not only by AMP but also by NAD⁺ (J. Biol. Chem. 2004, Dec. 17; 279 (51):52934-9). AMPK activated by phosphorylation has been reported to exhibit various functions and actions such as inhibition of fat synthesis, promotion of glucose uptake, promotion of fat degradation (lipolysis) and fat oxidation, promotion of glycolysis, enhancement of insulin sensitivity, suppression of glycogen synthesis, suppression of triglyceride and cholesterol synthesis, alleviation of inflammation (anti-inflammatory action), vasodilatory activity, functional improvement of cardiovascular systems, mitochondrial regeneration and muscle structural changes, anti-oxidative function, anti-aging and anti-cancer effects. In addition, due to the above-mentioned various activities and functions, AMPK is recognized as a target protein for treatments of diseases such as obesity, diabetes, metabolic syndromes, fatty liver, ischemic heart diseases, hypertension, degenerative cerebral diseases, hyperlipidemia, diabetic complications and erectile dysfunction (Nat. Med. 2004 July; 10(7):727-33; Nature reviews 3, 340-351, 2004; and Genes & Development 27, 1-6, 2004).

Lee, et al (Nature medicine, 13(June), 2004) have suggested that alpha-lipoic acid can exert anti-obesity effects by suppressing hypothalamic AMPK activity, thus controlling appetite. They have also reported that alpha-lipoic acid promotes fat metabolism via activation of AMPK in muscle tissues, not hypothalamus, and alpha-lipoic acid is therapeutically effective for the treatment of obesity because it facilitates energy expenditure by activating UCP-1, particularly in adipocytes.

Roger, et al (Cell, 117, 145-151, 2004) have suggested that an AMPK activating factor or a malonyl-CoA reducing factor may be a possible target to recover from or prevent such abnormal diseases and syndromes.

Nandakumar, et al (Progress in lipid research 42, 238-256, 2003) have proposed that, in ischemic heart diseases, AMPK would be a target to treat ischemia reperfusion injuries via regulation of fat and glucose metabolism.

Min, et al (Am. J. Physiol. Gastrointest Liver Physiol 287, G1-6, 2004) have reported that AMPK is effective for regulation of alcoholic fatty liver.

Genevieve, et al (J. Biol. Chem. 279, 20767-74, 2004) have reported that activation of AMPK inhibits activity of an iNOS enzyme which is an inflammation mediator in chronic inflammatory conditions or endotoxin shock, including obesity-related diabetes and thus AMPK will be effective for developing new medicines having a mechanism capable of enhancing insulin sensitivity. In addition, they have reported that inhibition of iNOS activity is effected by activation of AMPK, and thus this finding is clinically applicable to diseases such as septicemia, multiple sclerosis, myocardial infarction, inflammatory bowel diseases and pancreatic beta-cell dysfunction.

Zing-ping et al (FEBS Letters 443, 285-289, 1999) have reported that AMPK activates endothelial NO synthase through phosphorylation, in the presence of Ca-calmodulin in murine muscle cells and myocardial cells. This represents that AMPK is implicated in heart diseases including angina pectoris.

Javier, et al (Genes & Develop. 2004) have reported that a lifespan can be extended by limiting utilization of energy and such a prolonged lifespan is achieved in a manner that an in vivo AMP/ATP ratio is increased and therefore the α2 subunit of AMPK is activated by AMP. Therefore, they have suggested that AMPK may function as a sensor to detect the relationship between lifespan extension and energy level and insulin-like signal information.

Meanwhile, some pharmaceutical compositions having conventional naphthoquinone-based compounds as an active ingredient are known. Among them, β-lapachone is a naturally occurring plant product derived from lapachol obtained from the lapacho tree (Tabebuia avellanedae) which is native to South America. Dunnione and α-dunnione are also obtained from the leaves of Streptocarpus dunnii native to South America. Since ancient times in South America, these natural tricyclic naphthoquinone derivatives have been widely used as an anti-cancer drug and in the treatment of Chagas disease which is typically endemic in South America, and are also known to exert excellent therapeutic effects. In particular, as their pharmacological actions as the anti-cancer drug are generally known to western countries, these tricyclic naphthoquinone derivatives have lately attracted considerable attention from people. In fact, as disclosed in U.S. Pat. No. 5,969,163, such tricyclic naphthoquinone derivative compounds are currently developed as a variety of anti-cancer drugs by many research groups and institutions.

However, despite a variety of researches and studies, the fact that such naphthoquinone compounds have therapeutic efficacy for treating or preventing restenosis associated with surgical operations for arteriosclerosis or a treatment thereof caused by vascular smooth muscle cell proliferation via increasing NAD and AMPK activities still remains unknown.

### SUMMARY OF THE INVENTION

As a result of a variety of extensive and intensive studies and experiments based on the facts as described above, the inventors of the present invention have newly confirmed that activation of NQO1 via the use of specific compounds is effective for prevention and/or treatment of restenosis associated with surgical operations for a treatment of arteriosclerosis by inhibiting vascular smooth muscle cell proliferation. The present invention has been completed based on these findings.

Therefore, an object of the present invention is to provide a pharmaceutical composition comprising, as an active ingredient, a compound which is therapeutically effective for the treatment and prevention of restenosis associated with surgical operations for a treatment of arteriosclerosis.

In accordance with an aspect of the present invention, the above and other objects can be accomplished by the provision of a pharmaceutical composition for the treatment and/or prevention of restenosis, comprising: (a) a therapeutically effective amount of one or more compounds selected from compounds represented by Formula 1 and Formula 2 below, or a pharmaceutically acceptable salt, prodrug, solvate or isomer thereof:

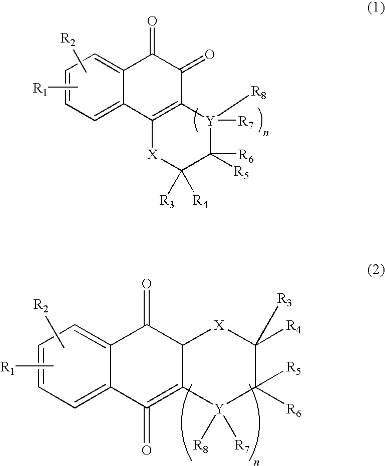

wherein

R₁ and R₂ are each independently hydrogen, halogen, amino, alkoxy, or C₁-C₆ lower alkyl or alkoxy, or R₁ and R₂ may be taken together to form a substituted or unsubstituted cyclic structure which may be saturated or partially or completely unsaturated;

R₃, R₄, R₅, R₆, R₇ and R₈ are each independently hydrogen, hydroxyl, amino, C₁-C₂₀ alkyl, alkene or alkoxy, C₄-C₂₀ cycloalkyl, heterocycloalkyl, aryl or heteroaryl, or two substituents of R₃ to R₈ may be taken together to form a cyclic structure which may be saturated or partially or completely unsaturated;

X is selected from a group consisting of C(R)(R′), N(R″), O and S, preferably O or S, and more preferably O, wherein R′ is hydrogen or C₁-C₆ lower alkyl;

Y is C, S or N, with proviso that when Y is S, R₇ and R₈ are nothing and when Y is N, R₇ is hydrogen or C₁-C₆ lower alkyl and R₈ is nothing; and

n is 0 or 1, with proviso that when n is 0, carbon atoms adjacent to n form a cyclic structure via a direct bond; and

(b) a pharmaceutically acceptable carrier, a diluent or an excipient, or any combination thereof.

In order to confirm therapeutic effects of the compound of Formula 1 on restenosis, the inventors of the present invention have conducted experiments. And, as a result, it was confirmed that when the compound according to Formula 1 or 2 was administered to vascular smooth muscle cell, it elevated NQO1 expression, and NAD⁺, which increased in accordance with the NQO1 expression, elevated AMPK activity to inhibit vascular smooth muscle cell proliferation. In addition, it was also confirmed that administration of the compound inhibited intimal hyperplasia after performing balloon angioplasty for treatment of arteriosclerosis.

In this regard, the compound of Formula 1 or 2 is believed to have excellent effects on the prevention and treatment of restenosis associated with vascular smooth muscle cell proliferation generated frequently after performing balloon angioplasty and arteriosclerosis associated with vascular smooth muscle cell proliferation after vessel wall injury by lipids.

As used the present disclosure, the term “pharmaceutically acceptable salt” means a formulation of a compound that does not cause significant irritation to an organism to which it is administered and does not abrogate the biological activity and properties of the compound. Examples of the pharmaceutical salt may include acid addition salts of the compound with acids capable of forming a non-toxic acid addition salt containing pharmaceutically acceptable anions, for example, inorganic acids such as hydrochloric acid, sulfuric acid, nitric acid, phosphoric acid, hydrobromic acid and hydroiodic acid; organic carbonic acids such as tartaric acid, formic acid, citric acid, acetic acid, tichloroacetic acid, trifluoroacetic acid, gluconic acid, benzoic acid, lactic acid, fumaric acid, maleic acid and salicylic acid; or sulfonic acids such as methanesulfonic acid, ethanesulfonic acid, benzenesulfonic acid and p-toluenesulfonic acid. Specifically, examples of pharmaceutically acceptable carboxylic acid salts include salts with alkali metals or alkaline earth metals such as lithium, sodium, potassium, calcium and magnesium, salts with amino acids such as arginine, lysine and guanidine, salts with organic bases such as dicyclohexylamine, N-methyl-D-tris(hydroxymethyl)methylamine, diethanolamine, choline and triethylamine. The compounds in accordance with the present invention may be converted into salts thereof, by conventional methods well-known in the art.

As used herein, the term “prodrug” means an agent that is converted into the parent drug in vivo. Prodrugs are often useful because, in some situations, they may be easier to administer than the parent drug. They may, for instance, be bioavailable by oral administration, whereas the parent may be not. The prodrugs may also have improved solubility in pharmaceutical compositions over the parent drug. An example of a prodrug, without limitation, would be a compound of the present invention which is administered as an ester (the “prodrug”) to facilitate transport across a cell membrane where water-solubility is detrimental to mobility, but which then is metabolically hydrolyzed to the carboxylic acid, the active entity, once inside the cell where water solubility is beneficial. A further example of the prodrug might be a short peptide (polyamino acid) bonded to an acidic group, where the peptide is metabolized to reveal the active moiety.

As an example of such prodrug, the pharmaceutical compounds in accordance with the present invention can include a prodrug represented by Formula 1a below as an active material:

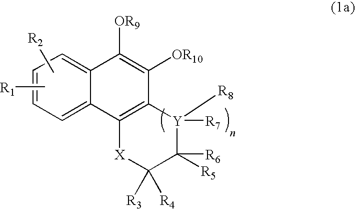

wherein,

R₁, R₂, R₃, R₄, R₅, R₆, R₇, R₈, X and n are as defined in Formula 1.

R₉ and R₁₀ are each independently —SO₃⁻Na⁺ or substituent represented by Formula A below or a salt thereof,

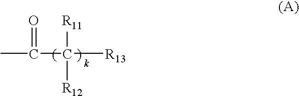

wherein,
R₁₁ and R₁₂ are each independently hydrogen or substituted or unsubstituted C₁-C₂₀ linear alkyl or C₁-C₂₀ branched alkyl,
R₁₃ is selected from the group consisting of substituents i) to viii) below:
i) hydrogen;
ii) substituted or unsubstituted C₁-C₂₀ linear alkyl or C₁-C₂₀ branched alkyl;
iii) substituted or unsubstituted amine;
iv) substituted or unsubstituted C₃-C₁₀ cycloalkyl or C₃-C₁₀ heterocycloalkyl;
v) substituted or unsubstituted C₄-C₁₀ aryl or C₄-C₁₀ heteroaryl;
vi) —(CRR′—NR″CO)ₗ—R₁₄, wherein R, R′ and R″ are each independently hydrogen or substituted or unsubstituted C₁-C₂₀ linear alkyl or C₁-C₂₀ branched alkyl, R₁₄ is selected from the group consisting of hydrogen, substituted or unsubstituted amine, cycloalkyl, heterocycloalkyl, aryl and heteroaryl, 1 is selected from the 1˜5;
vii) substituted or unsubstituted carboxyl;
viii) —OSO₃—Na⁺;
k is selected from the 0˜20, with proviso that when k is 0, R₁₁ and R₁₂ are not anything, and R₁₃ is directly bond to a carbonyl group.

As used herein, the term “solvate” means a compound of the present invention or a salt thereof, which further includes a stoichiometric or non-stoichiometric amount of a solvent bound thereto by non-covalent intermolecular forces. Preferred solvents are volatile, non-toxic, and/or acceptable for administration to humans. Where the solvent is water, the solvate refers to a hydrate.

As used herein, the term “isomer” means a compound of the present invention or a salt thereof that has the same chemical formula or molecular formula but is optically or sterically different therefrom. D type optical isomer and L type optical isomer can be present in the Formula 1, depending on the R₃˜R₈ types of substituents selected.

Unless otherwise specified, the term “compound of Formula 1 or 2” is intended to encompass a compound per se, and a pharmaceutically acceptable salt, prodrug, solvate and isomer thereof.

As used herein, the term “alkyl” refers to an aliphatic hydrocarbon group. The alkyl moiety may be a “saturated alkyl” group, which means that it does not contain any alkene or alkyne moieties. Alternatively, the alkyl moiety may also be an “unsaturated alkyl” moiety, which means that it contains at least one alkene or alkyne moiety. The term “alkene” moiety refers to a group in which at least two carbon atoms form at least one carbon-carbon double bond, and an “alkyne” moiety refers to a group in which at least two carbon atoms form at least one carbon-carbon triple bond. The alkyl moiety, regardless of whether it is substituted or unsubstituted, may be branched, linear or cyclic.

As used herein, the term “heterocycloalkyl” means a carbocyclic group in which one or more ring carbon atoms are substituted with oxygen, nitrogen or sulfur and which includes, for example, but is not limited to furan, thiophene, pyrrole, pyrroline, pyrrolidine, oxazole, thiazole, imidazole, imidazoline, imidazolidine, pyrazole, pyrazoline, pyrazolidine, isothiazole, triazole, thiadiazole, pyran, pyridine, piperidine, morpholine, thiomorpholine, pyridazine, pyrimidine, pyrazine, piperazine and triazine.

As used herein, the term “aryl” refers to an aromatic substituent group which has at least one ring having a conjugated pi (π) electron system and includes both carbocyclic aryl (for example, phenyl) and heterocyclic aryl(for example, pyridine) groups. This term includes monocyclic or fused-ring polycyclic (i.e., rings which share adjacent pairs of carbon atoms) groups.

As used herein, the term “heteroaryl” refers to an aromatic group that contains at least one heterocyclic ring.

Examples of aryl or heteroaryl include, but are not limited to, phenyl, furan, pyran, pyridyl, pyrimidyl and triazyl.

R₁, R₂, R₃, R₄, R₅, R₆, R₇ and R₈ in Formula 1 or 2 in accordance with the present invention may be optionally substituted. When substituted, the substituent group(s) is(are) one or more group(s) individually and independently selected from cycloalkyl, aryl, heteroaryl, heteroalicyclic, hydroxy, alkoxy, aryloxy, mercapto, alkylthio, arylthio, cyano, halogen, carbonyl, thiocarbonyl, O-carbamyl, N carbamyl, 0-thiocarbamyl, N-thiocarbamyl, C-amido, N-arnido, S-sulfonamido, N-sulfonamido, C-carboxy, O-carboxy, isocyanato, thiocyanato, isothiocyanato, nitro, silyl, trihalomethanesulfonyl, and amino including mono and di substituted amino, and protected derivatives thereof. Further, substituents of R₁₁, R₁₂ and R₁₃ in the Formula 1a may be also substituted as defined in above, and when substituted, they can be substituted as the substituents mentioned above.

Among compounds of Formula 1, preferred are compounds of Formulas 3 and 4 below.

Compounds of Formula 3 are compounds wherein n is 0 and adjacent carbon atoms form a cyclic structure (furan ring) via a direct bond therebetween and are often referred to as “furan compounds” or “furano-o-naphthoquinone derivatives” hereinafter.

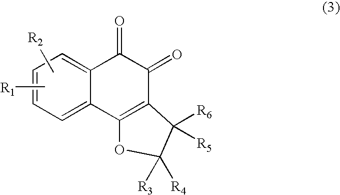

Compounds of Formula 4 are compounds wherein n is 1 and are often referred to as “pyran compounds” or “pyrano-o-naphthoquinone” hereinafter.

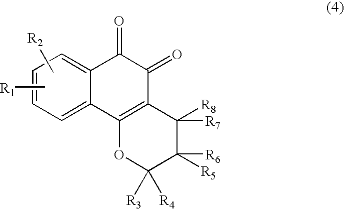

In Formula 1, each of R₁ and R₂ is particularly preferably hydrogen.

Among the furan compounds of Formula 3, particularly preferred are compounds of Formula 3a wherein R₁, R₂ and R₄ are hydrogen, or compounds of Formula 3b wherein R₁, R₂ and R₆ are hydrogen.

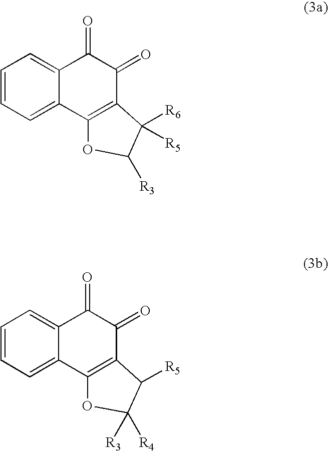

Further, among the pyran compounds of Formula 4, particularly preferred is compounds of Formula 4a wherein R₁, R₂, R₅, R₆, R₇ and R₈ are hydrogen or compounds of Formula 4b or 4c wherein R₁ and R₂ are taken together to form a cyclic structure which is substituted or unsubstituted.

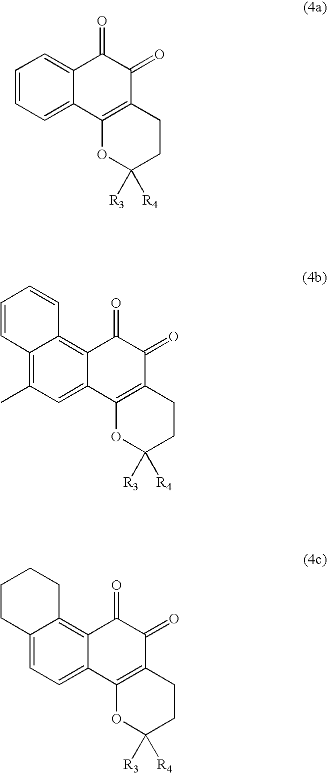

Among compounds of Formula 2, preferred without limitation, are compounds of Formulas 2a and 2b below.

Compounds of Formula 2a are compounds wherein n is 0 and adjacent carbon atoms form a cyclic structure via a direct bond therebetween and Y is C.

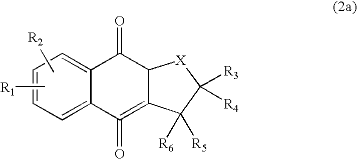

Compounds of Formula 2b are compounds wherein n is 1 and Y is C.

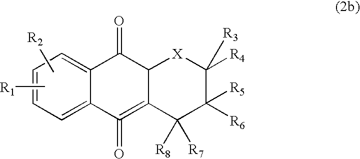

In the Formula 2a or 2b, R₁, R₂, R₃, R₄, R₅, R₆, R₇, R₈ and X are as defined in Formula 2.

The term “pharmaceutical composition” as used herein means a mixture of the compound of Formula 1 or 2 with other chemical components, such as diluents or carriers. The pharmaceutical composition facilitates administration of the compound to an organism. Various techniques of administering a compound are known in the art and include, but are not limited to oral, injection, aerosol, parenteral and topical administrations. Pharmaceutical compositions can also be obtained by reacting compounds of interest with acids such as hydrochloric acid, hydrobromic acid, sulfuric acid, nitric acid, phosphoric acid, methanesulfonic acid, p-toluenesulfonic acid, salicylic acid and the like. The effective ingredients, therapeutically effective for the treatment and prevention of restenosis include all the compounds of Formula in the above, referring “active ingredient” hereafter.

The term “therapeutically effective amount” means an amount of an active ingredient that is effective to relieve or reduce to some extent one or more of the symptoms of the disease in need of treatment, or to retard initiation of clinical markers or symptoms of a disease in need of prevention, when the compound is administered. Thus, a therapeutically effective amount refers to an amount of the active ingredient which exhibit effects of (i) reversing the rate of progress of a disease; (ii) inhibiting to some extent further progress of the disease; and/or, (iii) relieving to some extent (or, preferably, eliminating) one or more symptoms associated with the disease. The therapeutically effective amount may be empirically determined by experimenting with the compounds concerned in known in vivo and in vitro model systems for a disease in need of treatment.

### Preparation of Active Ingredient

In the pharmaceutical composition in accordance with the present invention, compounds of Formula 1 or 2 as an active ingredient, as will be illustrated hereinafter, can be prepared by conventional methods known in the art and/or various processes which are based upon the general technologies and practices in the organic chemistry synthesis field. The preparation processes described below are only exemplary ones and other processes can also be employed. As such, the scope of the instant invention is not limited to the following processes.

### Preparation Method 1: Synthesis of Active Materials by Acid-catalyzed Cyclization

Tricyclic naphthoquinone (pyrano-o-naphthoquinone and furano-o-naphthoquinone) derivatives having a relatively simple chemical structure are generally synthesized in a relatively high yield via cyclization using sulfuric acid as a catalyst, Based on this process, a variety of compounds of Formula 1 can be synthesized.

More specifically, the above synthesis process may be summarized as follows.

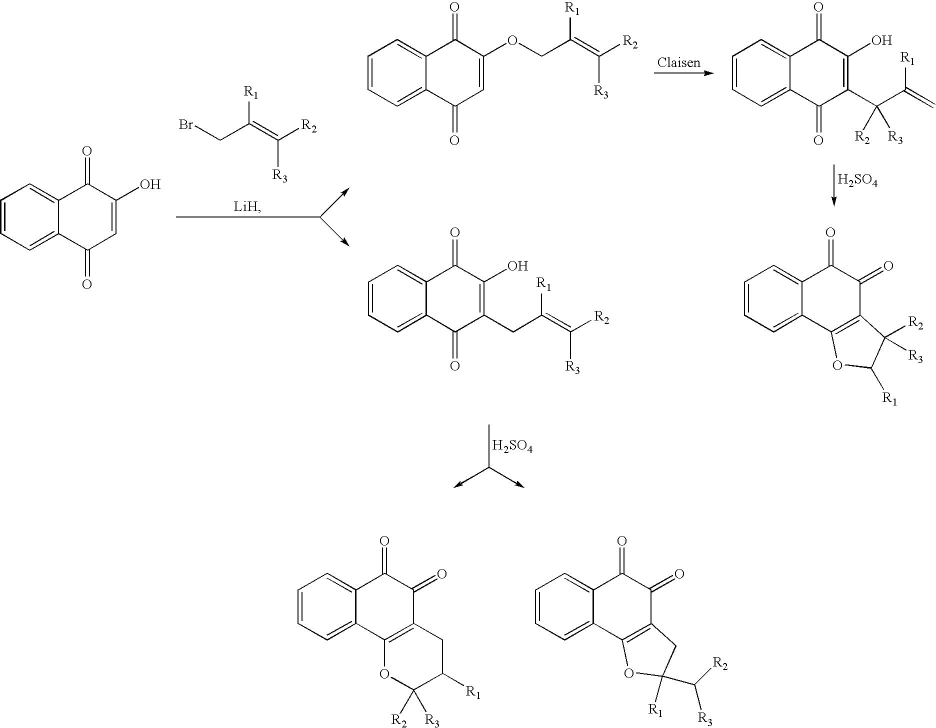

That is, when 2-hydroxy-1,4-naphthoquinone is reacted with various allylic bromides or equivalents thereof in the presence of a base, a C-alkylation product and an O-alkylation product are concurrently obtained. It is also possible to synthesize either of two derivatives only depending upon reaction conditions. Since O-alkylated derivative is converted into another type of C-alkylated derivative through Claisen Rearrangement by refluxing the O-alkylated derivative using a solvent such as toluene or xylene, it is possible to obtain various types of 3-substituted-2-hydroxy-1,4-naphthoquinone derivatives. The various types of C-alkylated derivatives thus obtained may be subjected to cyclization using sulfuric acid as a catalyst, thereby being capable of synthesizing pyrano-o-naphthoquinone or furano-o-naphthoquinone derivatives among compounds of Formula 1.

Preparation Method 2: Diels-Alder Reaction Using 3-methylene-1,2,4-[3H]naphthalenetione

As taught by V. Nair et al, Tetrahedron Lett. 42 (2001), 4549-4551, it is reported that a variety of pyrano-o-naphthoquinone derivatives can be relatively easily synthesized by subjecting 3-methylene-1,2,4-[3H]naphthalenetione, produced upon heating 2-hydroxy-1,4-naphthoquinone and formaldehyde together, to Diels-Alder reaction with various olefin compounds. This method is advantageous in that various forms of pyrano-o-naphtho-quinone derivatives can be synthesized in a relatively simplified manner, as compared to induction of cyclization using sulfuric acid as a catalyst.

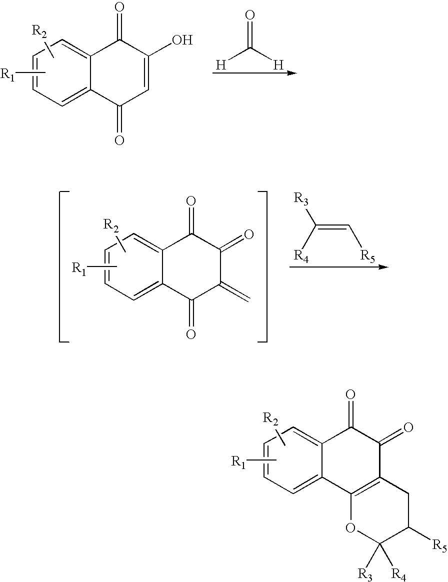

### Preparation Method 3: Haloakylation and Cyclization by Radical Reaction

The same method used in synthesis of Cryptotanshinone and 15,16-dihydro-tanshinone can also be conveniently employed for synthesis of furano-o-naphthoquinone derivatives. That is, as taught by A. C. Baillie et al (J. Chem. Soc. (C) 1968, 48-52), 2-haloethyl or 3-haloethyl radical chemical species, derived from 3-halopropanoic acid or 4-halobutanoic acid derivative, can be reacted with 2-hydroxy-1,4-naphthoquinone to thereby synthesize 3-(2-haloethyl or 3-halopropyl)-2-hydroxy-1,4-naphthoquinone which is then subjected to cyclization under suitable acidic catalyst conditions to synthesize various pyrano-o-naphthoquinone or furano-o-naphthoquinone derivatives.

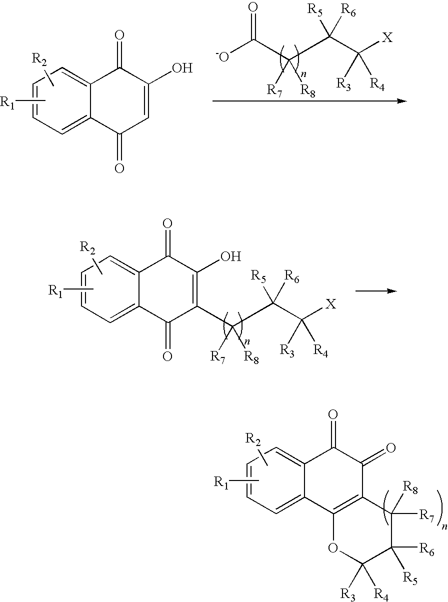

Preparation Method 4: Cyclization of 4,5-benzofurandione by Diels-Alder Reaction

Another method used in synthesis of Cryptotanshinone and 15,16-dihydro-tanshinone may be a method taught by J. K. Snyder et al (Tetrahedron Letters 28 (1987), 3427-3430). According to this method, furano-o-naphthoquinone derivatives can be synthesized by cycloaddition via Diels-Alder reaction between 4,5-benzofurandione derivatives and various diene derivatives.

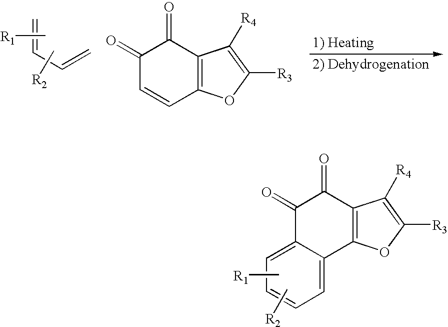

In addition, based on the above-mentioned preparation methods, various derivatives may be synthesized using relevant synthesis methods, depending upon kinds of substituents. Specific examples of derivatives thus synthesized and methods are exemplified in Table 1 below. Specific preparation methods will be described in the following Examples.

TABLE 1

| | | | | |
|---|---|---|---|---|
| 1 | 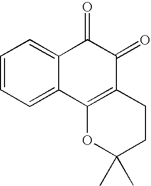 | C₁₅H₁₄O₃ | 242.27 | Method 1 |
| 2 | 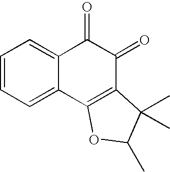 | C₁₅H₁₄O₃ | 242.27 | Method 1 |
| 3 | 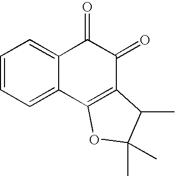 | C₁₅H₁₄O₃ | 242.27 | Method 1 |
| 4 | 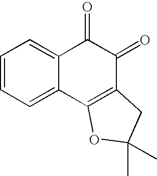 | C₁₄H₁₂O₃ | 228.24 | Method 1 |
| 5 | 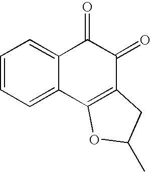 | C₁₃H₁₀O₃ | 214.22 | Method 1 |
| 6 | 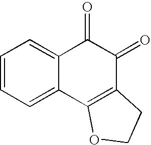 | C₁₂H₈O₃ | 200.19 | Method 2 |
| 7 | 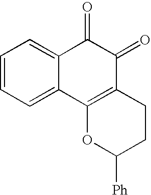 | C₁₉H₁₄O₃ | 290.31 | Method 1 |
| 8 | 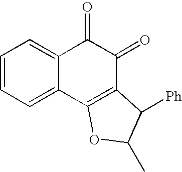 | C₁₉H₁₄O₃ | 290.31 | Method 1 |
| 9 | 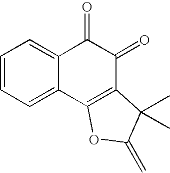 | C₁₅H₁₂O₃ | 240.25 | Method 1 |
| 10 | 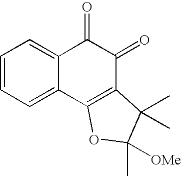 | C₁₆H₁₆O₄ | 272.30 | Method 1 |
| 11 | 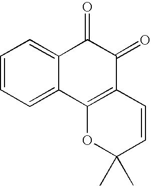 | C₁₅H₁₂O₃ | 240.25 | Method 1 |
| 12 | 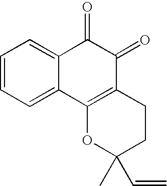 | C₁₆H₁₄O₃ | 254.28 | Method 2 |
| 13 | 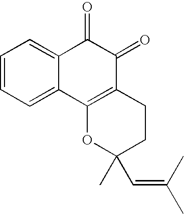 | C₁₈H₁₈O₃ | 282.33 | Method 2 |
| 14 | 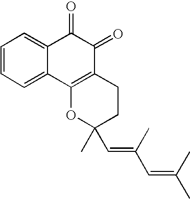 | C₂₁H₂₂O₃ | 322.40 | Method 2 |
| 15 | 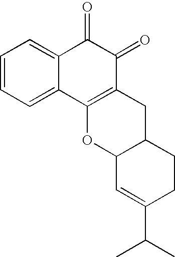 | C₂₁H₂₂O₃ | 322.40 | Method 2 |
| 16 | 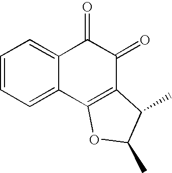 | C₁₄H₁₂O₃ | 228.24 | Method 1 |
| 17 | 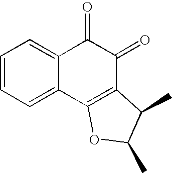 | C₁₄H₁₂O₃ | 228.24 | Method 1 |
| 18 | 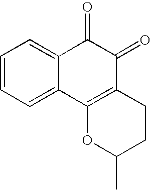 | C₁₄H₁₂O₃ | 228.24 | Method 1 |
| 19 | 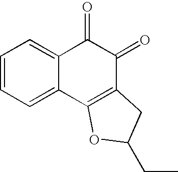 | C₁₄H₁₂O₃ | 228.24 | Method 1 |
| 20 | 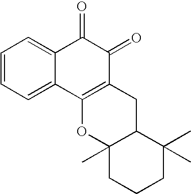 | C₂₀H₂₂O₃ | 310.39 | Method 1 |
| 21 | 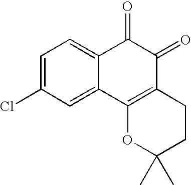 | C₁₅H₁₃ClO₃ | 276.71 | Method 1 |
| 22 | 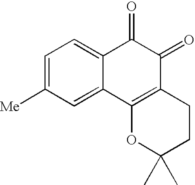 | C₁₆H₁₆O₃ | 256.30 | Method 1 |
| 23 | 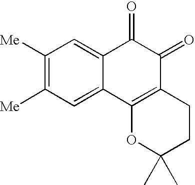 | C₁₇H₁₈O₅ | 302.32 | Method 1 |
| 24 | 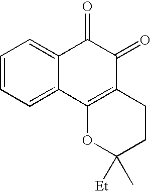 | C₁₆H₁₆O₃ | 256.30 | Method 1 |
| 25 | 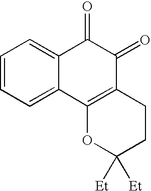 | C₁₇H₁₈O₃ | 270.32 | Method 1 |
| 26 | 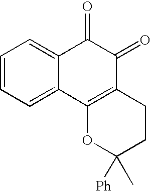 | C₂₀H₁₆O₃ | 304.34 | Method 1 |
| 27 | 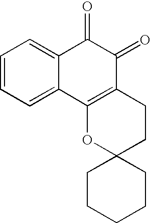 | C₁₈H₁₈O₃ | 282.33 | Method 1 |
| 28 | 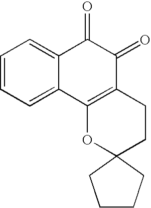 | C₁₇H₁₆O₃ | 268.31 | Method 1 |
| 29 | 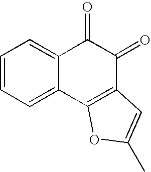 | C₁₃H₈O₃ | 212.20 | Method 1 |
| 30 | 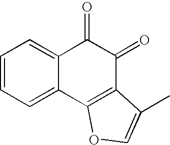 | C₁₃H₈O₃ | 212.20 | Method 4 |
| 31 | 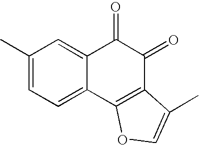 | C₁₄H₁₀O₃ | 226.23 | Method 4 |
| 32 | 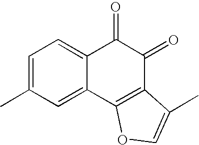 | C₁₄H₁₀O₃ | 226.23 | Method 4 |
| 33 | 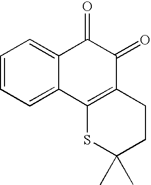 | C₁₅H₁₄O₂S | 258.34 | Method 1 |
| 34 | 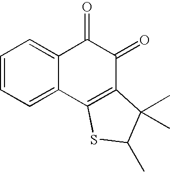 | C₁₅H₁₄O₂S | 258.34 | Method 1 |
| 35 | 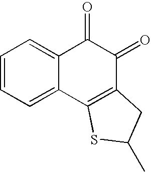 | C₁₃H₁₀O₂S | 230.28 | Method 1 |
| 36 | 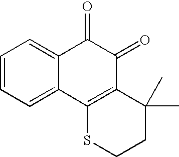 | C₁₅H₁₄O₂S | 258.34 | Method 2 |
| 37 | 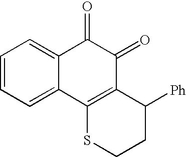 | C₁₉H₁₄O₂S | 306.38 | Method 2 |
| 38 | 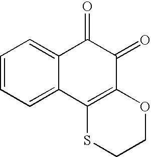 | C₁₂H₈O₃S | 232.26 | Method 3 |
| 39 | 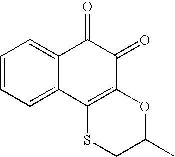 | C₁₃H₁₀O₃S | 246.28 | Method 3 |
| 40 | 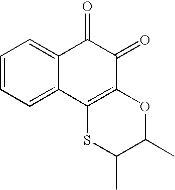 | C₁₄H₁₂O₃S | 260.31 | Method 3 |
| 41 | 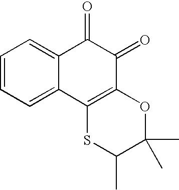 | C₁₅H₁₄O₃S | 274.34 | Method 3 |
| 42 | 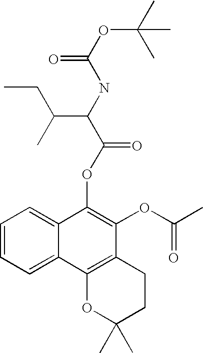 | C₂₈H₃₇O₇N | 502.22 | — |
| 43 | 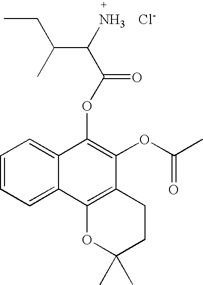 | C₂₃H₃₀O₅NCl | 940.32 | — |
| 44 | 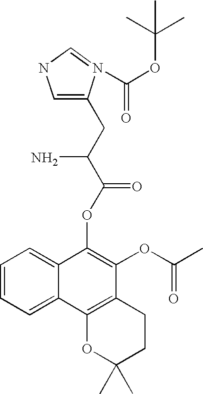 | C₂₈H₃₃O₇N₃ | 526.22 | — |
| 45 | 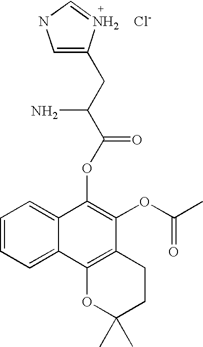 | C₂₃H₂₆O₅N₃Cl | 988.32 | — |
| 46 | 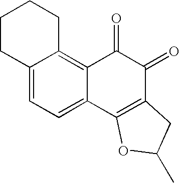 | C₁₇H₁₆O₃ | 268.31 | — |
| 47 | 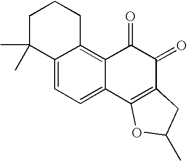 | C₁₉H₂₀O₃ | 296.36 | — |
| 48 | 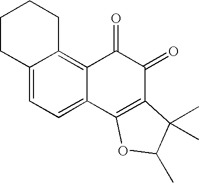 | C₁₉H₂₀O₃ | 296.36 | — |
| 49 | 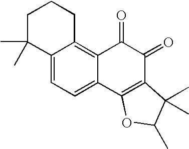 | C₂₁H₂₄O₃ | 324.41 | — |
| 50 | 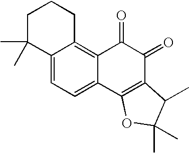 | C₂₁H₂₄O₃ | 324.41 | — |
| 51 | 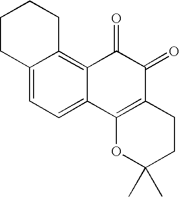 | C₁₉H₂₀O₃ | 296.36 | — |
| 52 | 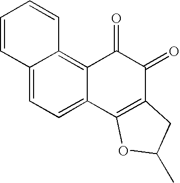 | C₁₇H₁₂O₃ | 264.28 | — |
| 53 | 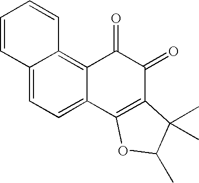 | C₁₉H₁₆O₃ | 292.33 | — |
| 54 | 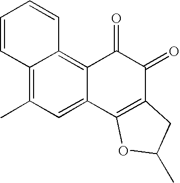 | C₁₈H₁₄O₃ | 278.30 | — |
| 55 | 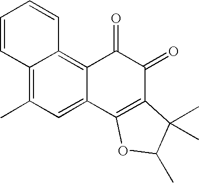 | C₂₀H₁₈O₃ | 306.36 | — |
| 56 | 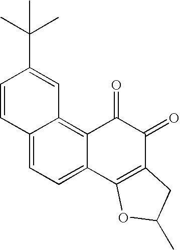 | C₂₁H₂₀O₃ | 320.38 | — |
| 57 | 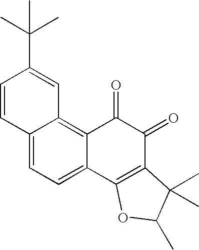 | C₂₃H₂₄O₃ | 348.43 | — |
| 58 | 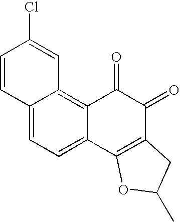 | C₁₇H₁₁ClO₃ | 298.72 | — |
| 59 | 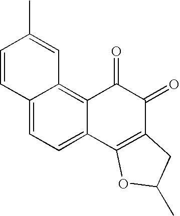 | C₁₈H₁₄O₃ | 278.30 | — |
| 60 | 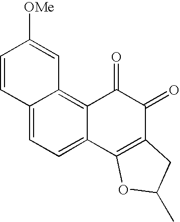 | C₁₈H₁₄O₄ | 294.30 | — |
| 61 | 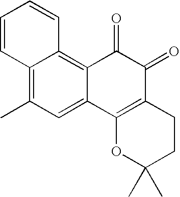 | C₂₀H₁₈O₃ | 306.36 | — |
| 62 | 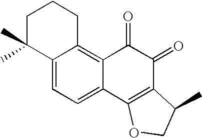 | C₁₈H₁₈O₃ | 282.33 | — |
| 63 | 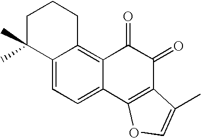 | C₁₈H₁₆O₃ | 280.33 | — |
| 64 | 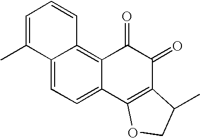 | C₁₈H₁₄O₃ | 278.33 | — |
| 65 | 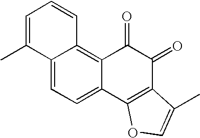 | C₁₈H₁₂O₃ | 276.33 | — |

The pharmaceutical composition of the present invention may be manufactured in a manner that is itself known, e.g., by means of conventional mixing, dissolving, granulating, dragee-making, levigating, emulsifying, encapsulating, entrapping or lyophilizing processes.

The pharmaceutical composition of the present invention may include additionally a pharmaceutically acceptable carrier, a diluent or an excipient, or any combination thereof. Pharmaceutical compositions for use in accordance with the present invention thus may be formulated in a conventional manner using one or more pharmaceutically acceptable carriers comprising excipients and auxiliaries which facilitate processing of the active compounds into preparations which can be used pharmaceutically. The pharmaceutical composition facilitates administration of the compound to an organism.

The term “carrier” means a chemical compound that facilitates the incorporation of a compound into cells or tissues. For example, dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the alt. One commonly used buffer solution is phosphate buffered saline (PBS) because it mimics the ionic strength conditions of human body fluid. Since buffer salts can control the pH of a solution at low concentrations, a buffer diluent rarely modifies the biological activity of a compound.

The compounds described herein may be administered to a human patient per se, or in the form of pharmaceutical compositions in which they are mixed with other active ingredients, as in combination therapy, or suitable carriers or excipient(s). Techniques for formulation and administration of the compounds may be found in “Remington's Pharmaceutical Sciences,” Mack Publishing Co., Easton, Pa., 18th edition, 1990.

Various techniques of administering a compound are known in the art and include, but are not limited to oral, injection, aerosol, parenteral and topical administrations. Pharmaceutical compositions can also be obtained by reacting compounds of interest with acids such as hydrochloric acid, hydrobromic acid, sulfuric acid, nitric acid, phosphoric acid, methanesulfonic acid, p-toluenesulfonic acid, salicylic acid and the like.

The compounds may be formulated by a variety of methods known in the art, preferably formulated into pharmaceutically acceptable oral, external, transmucosal and injectable preparation which is pharmaceutically acceptable, more preferably formulated into oral preparation.

The pharmaceutical composition of the present invention for oral administration is preferably prepared into intestine-targeted formulation.

Generally, an oral pharmaceutical composition passes through the stomach upon oral administration, is largely absorbed by the small intestine and then diffused into all the tissues of the body, thereby exerting therapeutic effects on the target tissues.

In this connection, the oral pharmaceutical composition according to the present invention enhances bioabsorption and bioavailability of the compound of Formula 1 or 2 as an active ingredient via intestine-targeted formulation of the active ingredient. More specifically, when the active ingredient in the pharmaceutical composition according to the present invention is primarily absorbed in the stomach, and upper parts of the small intestine, the active ingredient absorbed into the body directly undergoes liver metabolism which is then accompanied by substantial degradation of the active ingredient, so it is impossible to exert a desired level of therapeutic effects. On the other hand, it is expected that when the active ingredient is largely absorbed around and downstream of the lower small intestine, the absorbed active ingredient migrates via lymph vessels to the target tissues to thereby exert high therapeutic effects.

Further, as it is constructed in such a way that the pharmaceutical composition according to the present invention targets up to the colon which is a final destination of the digestion process, it is possible to increase the in vivo retention time of the drug and it is also possible to minimize decomposition of the drug which may take place due to the body metabolism upon administration of the drug into the body. As a result, it is possible to improve pharmacokinetic properties of the drug to significantly lower a critical effective dose of the active ingredient necessary for the treatment of the disease, and to obtain desired therapeutic effects even with administration of a trace amount of the active ingredient. Further, in the oral pharmaceutical composition, it is also possible to minimize the absorption variation of the drug by reducing the between- and within-individual variation of the bioavailability, which may result from intragastric pH changes and dietary uptake patterns.

Therefore, the intestine-targeted formulation according to the present invention is configured such that the active ingredient is largely absorbed in the small and large intestines, more preferably in the jejunum, and the ileum and colon corresponding to the lower small intestine, particularly preferably in the ileum or colon.

The intestine-targeted formulation may be designed by taking advantage of numerous physiological parameters of the digestive tract, through a variety of methods. In one preferred embodiment of the present invention, the intestine-targeted formulation may be prepared by (1) a formulation method based on a pH-sensitive polymer, (2) a formulation method based on a biodegradable polymer which is decomposable by an intestine-specific bacterial enzyme, (3) a formulation method based on a biodegradable matrix which is decomposable by an intestine-specific bacterial enzyme, or (4) a formulation method which allows release of a drug after a given lag time, and any combination thereof.

Specifically, the intestine-targeted formulation (1) using the pH-sensitive polymer is a drug delivery system which is based on pH changes of the digestive tract. The pH of the stomach is in a range of 1 to 3, whereas the pH of the small and large intestines has a value of 7 or more, which is higher as compared to that of the stomach. Based on this fact, the pH-sensitive polymer may be used in order to ensure that the pharmaceutical composition reaches the lower intestinal parts without being affected by pH fluctuations of the digestive tract. Examples of the pH-sensitive polymer may include, but are not limited to, at least one selected from the group consisting of methacrylic acid-ethyl acrylate copolymer (Eudragit: Registered Trademark of Rohm Pharma GmbH), hydroxypropylmethyl cellulose phthalate (HPMCP) and a mixture thereof.

Preferably, the pH-sensitive polymer may be added by a coating process. For example, addition of the polymer may be carried out by mixing the polymer in a solvent to form an aqueous coating suspension, spraying the resulting coating suspension to form a film coating, and drying the film coating.

The intestine-targeted formulation (2) using the biodegradable polymer which is decomposable by the intestine-specific bacterial enzyme is based on the utilization of a degradative ability of a specific enzyme that can be produced by enteric bacteria. Examples of the specific enzyme may include azoreductase, bacterial hydrolase glycosidase, esterase, polysaccharidase, and the like.

When it is desired to design the intestine-targeted formulation using azoreductase as a target, the biodegradable polymer may be a polymer containing an azoaromatic linkage, for example, a copolymer of styrene and hydroxyethylmethacrylate (HEMA). When the polymer is added to the formulation containing the active ingredient, the active ingredient may be liberated into the intestine by reduction of an azo group of the polymer via the action of the azoreductase which is specifically secreted by enteric bacteria, for example, Bacteroides fragilis and Eubacterium limosum.

When it is desired to design the intestine-targeted formulation using glycosidase, esterase, or polysaccharidase as a target, the biodegradable polymer may be a naturally-occurring polysaccharide or a substituted derivative thereof. For example, the biodegradable polymer may be at least one selected from the group consisting of dextran ester, pectin, amylase, ethyl cellulose and a pharmaceutically acceptable salt thereof. When the polymer is added to the active ingredient, the active ingredient may be liberated into the intestine by hydrolysis of the polymer via the action of each enzyme which is specifically secreted by enteric bacteria, for example, Bifidobacteria and Bacteroides spp. These polymers are natural materials, and have an advantage of low risk of in vivo toxicity.

The intestine-targeted formulation (3) using the biodegradable matrix which is decomposable by an intestine-specific bacterial enzyme may be a form in which the biodegradable polymers are cross-linked to each other and are added to the active ingredient or the active ingredient-containing formulation. Examples of the biodegradable polymer may include naturally-occurring polymers such as chondroitin sulfate, guar gum, chitosan, pectin, and the like. The degree of drug release may vary depending upon the degree of cross-linking of the matrix-constituting polymer.

In addition to the naturally-occurring polymers, the biodegradable matrix may be a synthetic hydrogel based on N-substituted acrylamide. For example, there may be used a hydrogel synthesized by cross-linking of N-tert-butylacryl amide with acrylic acid or copolymerization of 2-hydroxyethyl methacrylate and 4-methacryloyloxyazobenzene, as the matrix. The cross-linking may be, for example an azo linkage as mentioned above, and the formulation may be a form where the density of cross-linking is maintained to provide the optimal conditions for intestinal drug delivery and the linkage is degraded to interact with the intestinal mucous membrane when the drug is delivered to the intestine.

Further, the intestine-targeted formulation (4) with time-course release of the drug after a lag time is a drug delivery system utilizing a mechanism that is allowed to release the active ingredient after a predetermined time irrespective of pH changes. In order to achieve enteric release of the active drug, the formulation should be resistant to the gastric pH environment, and should be in a silent phase for 5 to 6 hours corresponding to a time period taken for delivery of the drug from the body to the intestine, prior to release of the active ingredient into the intestine. The time-specific delayed-release formulation may be prepared by addition of the hydrogel prepared from copolymerization of polyethylene oxide with polyurethane.

Specifically, the delayed-release formulation may have a configuration in which the formulation absorbs water and then swells while it stays within the stomach and the upper digestive tract of the small intestine, upon addition of a hydrogel having the above-mentioned composition after applying the drug to an insoluble polymer, and then migrates to the lower part of the small intestine which is the lower digestive tract and liberates the drug, and the lag time of drug is determined depending upon a length of the hydrogel.

As another example of the polymer, ethyl cellulose (EC) may be used in the delayed-release dosage formulation. EC is an insoluble polymer, and may serve as a factor to delay a drug release time, in response to swelling of a swelling medium due to water penetration or changes in the internal pressure of the intestines due to a peristaltic motion. The lag time may be controlled by the thickness of EC. As an additional example, hydroxypropylmethyl cellulose (HPMC) may also be used as a retarding agent that allows drug release after a given period of time by thickness control of the polymer, and may have a lag time of 5 to 10 hours.

In the oral pharmaceutical composition according to the present invention, the active ingredient may have a crystalline structure with a high degree of crystallinity, or a crystalline structure with a low degree of crystallinity. Preferably, the active ingredient may have a crystalline structure with a low degree of crystallinity, which can solve the problems associated with sparingly-solubility in the compound of Formula 1 or 2, and increase the dissolution rate and in vivo absorption rate.

As used herein, the term “degree of crystallinity” is defined as the weight fraction of the crystalline portion of the total compound and may be determined by a conventional method known in the art. For example, measurement of the degree of crystallinity may be carried out by a density method or precipitation method which calculates the crystallinity degree by previous assumption of a preset value obtained by addition and/or reduction of appropriate values to/from each density of the crystalline portion and the amorphous portion, a method involving measurement of the heat of fusion, an X-ray method in which the crystallinity degree is calculated by separation of the crystalline diffraction fraction and the noncrystalline diffraction fraction from X-ray diffraction intensity distribution upon X-ray diffraction analysis, or an infrared method which calculates the crystallinity degree from a peak of the width between crystalline bands of the infrared absorption spectrum.

In the oral pharmaceutical composition according to the present invention, the crystallinity degree of the active ingredient is preferably 50% or less. More preferably, the active ingredient may have an amorphous structure from which the intrinsic crystallinity of the material was completely lost. The amorphous compound exhibits a relatively high solubility, as compared to the crystalline compound, and can significantly improve a dissolution rate and in vivo absorption rate of the drug.

In one preferred embodiment of the present invention, the amorphous structure may be formed during preparation of the active ingredient into microparticles or fine particles (micronization of the active ingredient). The microparticles may be prepared, for example by spray drying of active ingredients, melting methods involving formation of melts of active ingredients with polymers, co-precipitation involving formation of co-precipitates of active ingredients with polymers after dissolution of active ingredients in solvents, inclusion body formation, solvent volatilization, and the like. Preferred is spray drying. Even when the active ingredient is not of an amorphous structure, that is has a crystalline structure or semi-crystalline structure, micronization of the active ingredient into fine particles via mechanical milling contributes to improvement of solubility, due to a large specific surface area of the particles, consequently resulting in improved dissolution rate and bioabsorption rate of the active drug.

The spray drying is a method of making fine particles by dissolving the active ingredient in a certain solvent and the spray-drying the resulting solution. During the spray-drying process, a high percent of the crystallinity of the compound is lost to thereby result in an amorphous state, and therefore the spray-dried product in the form of a fine powder is obtained.

The mechanical milling is a method of grinding the active ingredient into fine particles by applying strong physical force to active ingredient particles. The mechanical milling may be carried out by using a variety of milling processes such as jet milling, ball milling, vibration milling, hammer milling, and the like. Particularly preferred is jet milling which can be carried out using an air pressure, at a temperature of less than 40° C.

Meanwhile, irrespective of the crystalline structure, a decreasing particle diameter of the particulate active ingredient leads to an increasing specific surface area, thereby increasing the dissolution rate and solubility. However, an excessively small particle diameter makes it difficult to prepare fine particles having such a size and also brings about agglomeration or aggregation of particles which may result in deterioration of the solubility. Therefore, in one preferred embodiment, the particle diameter of the active ingredient may be in a range of 5 nm to 500 μm. In this range, the particle agglomeration or aggregation can be maximally inhibited, and the dissolution rate and solubility can be maximized due to a high specific surface area of the particles.

Preferably, a surfactant may be additionally added to prevent the particle agglomeration or aggregation which may occur during formation of the fine particles, and/or an antistatic agent may be additionally added to prevent the occurrence of static electricity.

If necessary, a moisture-absorbent material may be further added during the milling process. The compound of Formula 1 or 2 has a tendency to be crystallized by water, so incorporation of the moisture-absorbent material inhibits recrystallization of the compound over time and enables maintenance of increased solubility of compound particles due to micronization. Further, the moisture-absorbent material serves to suppress coagulation and aggregation of the pharmaceutical composition while not adversely affecting therapeutic effects of the active ingredient.

Examples of the surfactant may include, but are not limited to, anionc surfactants such as docusate sodium and sodium lauryl sulfate; cationic surfactants such as benzalkonium chloride, benzethonium chloride and cetrimide; nonionic surfactants such as glyceryl monooleate, polyoxyethylene sorbitan fatty acid ester, and sorbitan ester; amphiphilic polymers such as polyethylene-polypropylene polymer and polyoxyethylene-polyoxypropylene polymer (Poloxamer), and Gelucire™ series (Gattefosse Corporation, USA); propylene glycol monocaprylate, oleoyl macrogol-6-glyceride, linoleoyl macrogol-6-glyceride, caprylocaproyl macrogol-8-glyceride, propylene glycol monolaurate, and polyglyceryl-6-dioleate. These materials may be used alone or in any combination thereof.

Examples of the moisture-absorbent material may include, but are not limited to, colloidal silica, light anhydrous silicic acid, heavy anhydrous silicic acid, sodium chloride, calcium silicate, potassium aluminosilicate, calcium aluminosilicate, and the like. These materials may be used alone or in any combination thereof.

Some of the above-mentioned moisture absorbents may also be used as the antistatic agent.

The surfactant, antistatic agent, and moisture absorbent are added in a certain amount that is capable of achieving the above-mentioned effects, and such an amount may be appropriately adjusted depending upon micronization conditions. Preferably, the additives may be used in a range of 0.05 to 20% by weight, based on the total weight of the active ingredient.

In one preferred embodiment, during formulation of the pharmaceutical composition according to the present invention into preparations for oral administration, water-soluble polymers, solubilizers and disintegration-promoting agents may be further added. Preferably, formulation of the composition into a desired dosage form may be made by mixing the additives and the particulate active ingredient in a solvent and spray-drying the mixture.

The water-soluble polymer is of help to prevent aggregation of the particulate active ingredients, by rendering surroundings of compound molecules or particles of Formula 1 or 2 hydrophilic to consequently enhance water solubility, and preferably to maintain the amorphous state of the naphtoquinone-based compound as an active ingredient.

Preferably, the water-soluble polymer is a pH-independent polymer, and can bring about crystallinity loss and enhanced hydrophilicity of the active ingredient, even under the between- and within-individual variation of the gastrointestinal pH.

Preferred examples of the water-soluble polymers may include at least one selected from the group consisting of cellulose derivatives such as methyl cellulose, hydroxymethyl cellulose, hydroxyethyl cellulose, ethyl cellulose, hydroxyethylmethyl cellulose, carboxymethyl cellulose, hydroxypropylmethyl cellulose, hydroxypropylmethyl cellulose phthalate, sodium carboxymethyl cellulose, and carboxymethylethyl cellulose; polyvinyl alcohols; polyvinyl acetate, polyvinyl acetate phthalate, polyvinylpyrrolidone (PVP), and polymers containing the same; polyalkene oxide or polyalkene glycol, and polymers containing the same. Preferred is hydroxypropylmethyl cellulose.

In the pharmaceutical composition of the present invention, an excessive content of the water-soluble polymer which is higher than a given level provides no further increased solubility, but disadvantageously brings about various problems such as overall increases in the hardness of the formulation, and non-penetration of an eluent into the formulation, by formation of films around the formulation due to excessive swelling of water-soluble polymers upon exposure to the eluent. Accordingly, the solubilizer is preferably added to maximize the solubility of the formulation by modifying physical properties of the compound of Formula 1 or 2.

In this respect, the solubilizer serves to enhance solubilization and wettability of the sparingly-soluble compound of Formula 1 or 2, and can significantly reduce the bioavailability variation of the compound of Formula 1 or 2 originating from diets and the time difference of drug administration after dietary uptake. The solubilizer may be selected from conventionally widely used surfactants or amphiphiles, and specific examples of the solubilizer may refer to the surfactants as defined above.

The disintegration-promoting agent serves to improve the drug release rate, and enables rapid release of the drug at the target site to thereby increase bioavailability of the drug.

Preferred examples of the disintegration-promoting agent may include, but are not limited to, at least one selected from the group consisting of Croscarmellose sodium, Crospovidone, calcium carboxymethylcellulose, starch glycolate sodium and lower substituted hydroxypropyl cellulose. Preferred is Croscarmellose sodium.

Upon taking into consideration various factors as described above, it is preferred to add 10 to 1000 parts by weight of the water-soluble polymer, 1 to 30 parts by weight of the disintegration-promoting agent and 0.1 to 20 parts by weight of the solubilizer, based on 100 parts by weight of the active ingredient.

In addition to the above-mentioned ingredients, other materials known in the art in connection with formulation may be optionally added, if necessary.

The solvent for spray drying is a material exhibiting a high solubility without modification of physical properties thereof and easy volatility during the spray drying process. Preferred examples of such a solvent may include, but are not limited to, dichloromethane, chloroform, methanol, and ethanol. These materials may be used alone or in any combination thereof. Preferably, a content of solids in the spray solution is in a range of 5 to 50% by weight, based on the total weight of the spray solution.

The above-mentioned intestine-targeted formulation process may be preferably carried out for formulation particles prepared as above.

In one preferred embodiment, the oral pharmaceutical composition according to the present invention may be formulated by a process comprising the following steps:

(a) adding the compound of Formula 1 or 2 alone or in combination with a surfactant and a moisture-absorbent material, and grinding the compound of Formula 1 or 2 with a jet mill to prepare active ingredient microparticles;

(b) dissolving the active ingredient microparticles in conjunction with a water-soluble polymer, a solubilizer and a disintegration-promoting agent in a solvent and spray-drying the resulting solution to prepare formulation particles; and

(c) dissolving the formulation particles in conjunction with a pH-sensitive polymer and a plasticizer in a solvent and spray-drying the resulting solution to carry out intestine-targeted coating on the formulation particles.

The surfactant, moisture-absorbent material, water-soluble polymer, solubilizer and disintegration-promoting agent are as defined above. The plasticizer is an additive added to prevent hardening of the coating, and may include, for example polymers such as polyethylene glycol.

Alternatively, formulation of the active ingredient may be carried out by sequential or concurrent spraying of vehicles of Step (b) and intestine-targeted coating materials of Step (c) onto jet-milled active ingredient particles of Step (a) as a seed.

For injection, the agents of the present invention may be formulated in aqueous solutions, preferably in physiologically compatible buffers such as Hanks's solution, Ringer's solution, or physiological saline. For transmucosal administration, penetrants appropriate to the barrier to be permeated are used in the formulation. Such penetrants are generally known in the art.

The compounds may be formulated for parenteral administration by injection, e.g., by bolus injection or continuous infusion. Formulations for injection may be presented in unit dosage forms, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Alternatively, the active ingredient may be in powder form for constitution with a suitable vehicle, e.g., sterile pyrogen-five water, before use.

The pharmaceutical composition in accordance with the present invention may be prepared or added into a common tablet form, as well as various forms capable of delivering the active ingredients to the disease region. For example, the composition may be added by being coated on or embedded in a mesh stent to be inserted in blood vessels. The stent is commonly inserted by a surgical operation to regulate blood or body fluid flow in vessels, gastrointestinal tracts, biliary tracts or the like. It is a mesh-like material made of a stainless steel, a shape memory alloy, nitinol (Ti—Ni) or the like. Therefore, the pharmaceutical composition in accordance with the present invention may be applied to the disease region through being directly coated on or attached via a predetermined binder to the outer surface of the stent, or embedded in the stent in a form capable of discharging outward. In the above, only the stent has been exemplified as a medium for adding the active ingredient, but those skilled in the art will appreciate that various modifications, additions and substitutions are possible, without departing from the scope and spirit of the invention.

Pharmaceutical compositions suitable for use in the present invention include compositions in which the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

When the pharmaceutical composition of the present invention is formulated into a unit dosage form, the compound of Formula 1 or 2 as the active ingredient is preferably contained in a unit dose of about 0.1 to 1,000 mg. The amount of the compound of Formula 1 or 2 administered will be determined by the attending physician, depending upon body weight and age of patients being treated, characteristic nature and the severity of diseases.

The present invention also provides use of the compound of Formula 1 or 2 in the preparation of a drug for preventing and treating of restenosis. As used herein, the term “treatment” refers to stopping or delaying of the disease progress, when the drug is used in the subject exhibiting symptoms of disease onset. The term “prevention” refers to stopping or delaying of symptoms of disease onset, when the drug is used in the subject exhibiting no symptoms of disease onset but having high risk of disease onset.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a bar graph showing counting results on numbers of living cells after pre-treatment with Compound 1 according to the present invention by varying the concentration;

FIG. 2 is a result of RT-PCR confirming expression of NQO1 in vascular smooth muscle cells treated with Compound 1 according to the present invention;

FIG. 3 is a result confirming a degree of phosphorylation of AMPK and ACC in vascular smooth muscle cells treated with Compound 1 according to the present invention;

FIG. 4 is a result confirming expressions of p53, p21, retinoblastoma and CDK in vascular smooth muscle cells treated with Compound 1 according to the present invention;

FIG. 5 is a result confirming inhibition of cell proliferation in vascular smooth muscle cells treated with Compound 1 according to the present invention;

FIG. 6 is an observation result of vessel intimal hyperplasia in rats with administration of Compound 1 according to the present invention; and

FIG. 7 is a comparison/observation result a degree of lipid accumulation on inner vessel wall and aortic valve after staining with Oil Red O by killing rats with administration of Compound 1 according to the present invention.

### DETAILED DESCRIPTION OF PREFERRED EMBODIMENTS

Now, the present invention will be described in more detail with reference to the following Examples. These examples are provided only for illustrating the present invention and should not be construed as limiting the scope and spirit of the present invention.

### Example 1

### Synthesis of β-lapachone (Compound 1)

17.4 g (0.10M) of 2-hydroxy-1,4-naphthoquinone was dissolved in 120 ml of DMSO, and 0.88 g (0.11M) of LiH was gradually added thereto. Here, this should be done with care because hydrogen evolves. The reaction solution was stirred, and after confirming no further production of hydrogen, was additionally stirred for another 30 min. Then, 15.9 g (0.10M) of prenyl bromide (1-bromo-3-methyl-2-butene) and 3.35 g (0.025M) of LiI were gradually added thereto. The reaction solution was heated to 45° C. and then stirred vigorously for 12 hours at that temperature. The reaction solution was cooled below 10° C., and 76 g of ice was first added and 250 ml of water was then added. Thereafter, 25 ml of concentrated HCl was gradually added to maintain the resulting solution at an acidic pH>1. 200 ml of EtOAc was added to the reaction mixture which was then stirred vigorously, thereby producing white solids that were not dissolved in EtOAc. These solids were filtered and an EtOAc layer was separated. The aqueous layer was extracted once again with 100 ml of EtOAc and was combined with the previously extracted organic layer. The organic layer was washed with 150 ml of 5% NaHCO₃, and was concentrated. The resulting concentrates were dissolved in 200 ml of CH₂Cl₂, and were vigorously shaken to separate two layers with addition of 70 ml of an aqueous 2N NaOH solution. A CH₂Cl₂ layer was further separated twice with treatment of an aqueous 2N NaOH solution (70 ml×2). The thus-separated aqueous solutions were combined together and adjusted to an acidic pH>2, thereby forming solids. The resulting solids were filtered and separated to give Lapachol. The thus-obtained Lapachol was recrystallized from 75% EtOH. The resulting Lapachol was mixed with 80 ml of sulfuric acid, and the mixture was vigorously stirred at mom temperature for 10 min and 200 g of ice was added thereto to complete the reaction. 60 ml of CH₂Cl₂ was added to the reaction materials which were then shaken vigorously. Thereafter, a CH₂Cl₂ layer was separated and washed with 5% NaHCO₃. An aqueous layer was extracted once again using 30 ml of CH₂Cl₂, washed with 5% NaHCO₃ and combined with the previously extracted organic layer. The organic layer was dried over MgSO₄ and concentrated to give impure β-Lapachone. The thus-obtained β-Lapachone was recrystallized from isopropanol, thereby obtaining 8.37 g of pure β-Lapachone.

¹H-NMR. (CDCl₃, 8): 8.05 (1H, dd, J=1, 8 Hz), 7.82 (1H, dd, J=1, 8 Hz), 7.64 (1H, dt, J=1, 8 Hz), 7.50 (1H, dt, J=1, 8 Hz), 2.57 (2H, t, J=6.5 Hz), 1.86 (2H, t, J=6.5 Hz) 1.47 (6H, s)

### Example 2

### Synthesis of Dunnione (Compound 2)

In the process of obtaining Lapachol in Example 1, solids separated without being dissolved in EtOAc are 2-prenyloxy-1,4-naphthoquinone, an O-akylation product, unlike Lapachol which is a C-alylation product. The separated 2-prenyloxy-1,4-naphthoquinone was first recrystallized once again from EtOAc. 3.65 g (0.015M) of the thus-purified solids was dissolved in toluene and toluene was refluxed for 5 hours to induce Claisen Rearrangement. Toluene was concentrated by distillation under reduced pressure and was then mixed with 15 ml of sulfuric acid, without further purification. The resulting mixture was stirred vigorously at room temperature for 10 min and 100 g of ice was added thereto to complete the reaction. 50 ml of CH₂Cl₂ was added to the reaction materials which were shaken vigorously. Thereafter, a CH₂Cl₂ layer was separated and washed with 5% NaHCO₃. An aqueous layer was extracted once again using 20 ml of CH₂Cl₂, washed with 5% NaHCO₃ and combined with the previously extracted organic layer. The organic layer was dried over MgSO₄, concentrated and purified by chromatography on silica gel to give 2.32 g of pure Dunnione.

¹H-NMR (CDCl₃, 8): 8.05 (1H, d, J=8 Hz), 7.64 (2H, d, J=8 Hz), 7.56 (1H, m), 4.67 (1H, q, J=7 Hz), 1.47 (3H, d, J=7 Hz), 1.45 (3H, s) 1.27 (3H, s)

### Example 3

### Synthesis of α-Dunnione (Compound 3)

4.8 g (0.020M) of 2-prenyloxy-1,4-naphthoquinone purified in Example 2 was dissolved in xylene, and xylene was refluxed for 15 hours, thereby inducing Claisen Rearrangement under significantly higher temperature conditions and prolonged reaction conditions as compared to Example 2. According to this reaction process, α-Dunnione that had progressed to cyclization was obtained together with a Lapachol derivative which had undergone Claisen Rearrangement and in which one of two methyl groups has shifted. Xylene was concentrated by distillation under reduced pressure and purified by chromatography on silica gel to give 1.65 g of pure α-Dunnione.

¹H-NMR (CDCl₃, δ): 8.06 (1H, d, J=8 Hz), 7.64 (2H, m), 7.57 (1H, m), 3.21 (1H, q, J=7 Hz), 1.53 (3H, s), 1.51 (3H, s) 1.28 (3H, d, J=7 Hz)

### Example 4

### Synthesis of Compound 4

17.4 g (0.10M) of 2-hydroxy-1,4-naphthoquinone was dissolved in 120 ml of DMSO, and 0.88 g (0.11M) of LiH was gradually added thereto. Here, this should be done with care because hydrogen evolves. The reaction solution was stirred, and after confirming no further production of hydrogen, was additionally stirred for another 30 min. Then, 14.8 g (0.11M) of methallyl bromide (1-bromo-2-methylpropene) and 3.35 g (0.025M) of Lil were gradually added thereto. The reaction solution was heated to 45° C. and then stirred vigorously for 12 hours at that temperature. The reaction solution was cooled below 10° C., and 80 g of ice was first added and 250 ml of water was then added. Thereafter, 25 ml of concentrated HCl was gradually added to maintain the resulting solution at an acidic pH>1. 200 ml of CH₂Cl₂ was added to the reaction mixture which was then shaken vigorously to separate two layers. The aqueous layer was extracted once again with addition of 70 ml of CH₂Cl₂ and was combined with the previously extracted organic layer. Two materials were confirmed to be formed newly by TLC and were subsequently used without any particular separation process. The organic layer was concentrated by distillation under reduced pressure, dissolved again in xylene and then refluxed for 8 hours. In this process, two materials on TLC were combined into one, thereby obtaining a relatively pure Lapachol derivative. The thus-obtained Lapachol derivative was mixed with 80 ml of sulfuric acid and stirred vigorously at room temperature for 10 min, and 200 g of ice was added thereto to complete the reaction. 80 ml of CH₂Cl₂ was added to the reaction materials which were then shaken vigorously. Thereafter, a CH₂Cl₂ layer was separated and washed with 5% NaHCO₃. An aqueous layer was extracted once again using 50 ml of CH₂Cl₂, washed with 5% NaHCO₃ and combined with the previously extracted organic layer. The organic layer was dried over MgSO₄ and concentrated to give impure (β-Lapachonederivative (Compound 4). The thus-obtained β-Lapachone derivative was recrystallized from isopropanol, thereby obtaining 12.21 g of pure Compound 4.

¹H-NMR (CDCl₃, δ): 8.08 (1H, d, J=8 Hz), 7.64 (2H, m), 7.57 (1H, m), 2.95 (2H, s), 1.61 (6H, s)

### Example 5

### Synthesis of Compound 5

Compound 5 was obtained in the same manner as in Example 4, except that allyl bromide was used instead of methallyl bromide.

¹H-NMR (CDCl₃, δ): 8.07 (1H, d, J=7 Hz), 7.65 (2H, m), 7.58 (1H, m), 5.27 (1H, m), 3.29 (1H, dd, J=10, 15 Hz), 2.75 (1H, dd, J=7, 15 Hz), 1.59 (3H, d, J=6 Hz)

### Example 6

### Synthesis of Compound 6

5.08 g (40 mM) of 3-chloropropionyl chloride was dissolved in 20 ml of ether and cooled to −78° C. 1.95 g (25 mM) of sodium peroxide (Na₂O₂) was gradually added to the resulting solution while being vigorously stirred at that temperature, followed by further vigorous stirring for 30 min. The reaction solution was heated to 0° C. and 7 g of ice was added thereto, followed by additional stirring for another 10 min. An organic layer was separated, washed once again with 10 ml of cold water at 0° C., then with an aqueous NaHCO₃ solution at 0° C. The organic layer was separated, dried over MgSO₄, concentrated by distillation under reduced pressure below 0° C., thereby preparing 3-chloropropionic peracid.

1.74 g (10 mM) of 2-hydroxy-1,4-naphthoquinone was dissolved in 20 ml of acetic acid, and the previously prepared 3-chloropropionic peracid was gradually added thereto at room temperature. The reaction mixture was refluxed with stirring for 2 hours, and then distilled under reduced pressure to remove acetic acid. The resulting concentrates were dissolved in 20 ml of CH₂Cl₂, and washed with 20 ml of 5% NaHCO₃. An aqueous layer was extracted once again using 20 ml of CH₂Cl₂ and combined with the previously extracted organic layer. The organic layer was dried over MgSO₄ and concentrated to give Compound 6 in admixture with 2-(2-chloroethyl)-3-hydroxy-1,4-naphthoquinone. The resulting mixture was purified by chromatography on silica gel to give 0.172 g of a pure Lapachone derivative (Compound 6).

¹H-NMR (CDCl₃, 8): 8.07 (1H, d, J=7.6 Hz), 7.56-7.68 (3H, m), 4.89 (2H, t, J=9.2 Hz), 3.17 (2H, t, J=9.2 Hz)

### Example 7

### Synthesis of Compound 7

17.4 g (0.10M) of 2-hydroxy-1,4-naphthoquinone was dissolved in 120 ml of DMSO, and 0.88 g (0.11M) of LiH was gradually added thereto. Here, this should be done with care because hydrogen evolves. The reaction solution was stirred, and after confirming no further production of hydrogen, was additionally stirred for another 30 min. Then, 19.7 g (0.10M) of cinnamyl bromide (3-phenylallyl bromide) and 3.35 g (0.025M) of LiI were gradually added thereto. The reaction solution was heated to 45° C. and then stirred vigorously for 12 hours at that temperature. The reaction solution was cooled below 10° C., and 80 g of ice was first added and 250 ml of water was then added. Thereafter, 25 ml of concentrated HCl was gradually added to maintain the resulting solution at an acidic pH>1.200 ml of CH₂Cl₂ was added to dissolve the reaction mixture which was then shaken vigorously to separate two layers. The aqueous layer was discarded, and a CH₂Cl₂ layer was treated with an aqueous 2N NaOH solution (100 mlx2) to separate the aqueous layer twice. At this time, the remaining CH₂Cl₂ layer after extraction with an aqueous 2N NaOH solution was used again in Example 8. The thus-separated aqueous solutions were combined and adjusted to an acidic pH>2 using concentrated HCl, thereby forming solids. The resulting solids were filtered and separated to give a Lapachol derivative. The thus-obtained Lapachol derivative was recrystallized from 75% EtOH. The resulting Lapachol derivative was mixed with 50 ml of sulfuric acid, and the mixture was vigorously stirred at room temperature for 10 min and 150 g of ice was added thereto to complete the reaction. 60 ml of CH₂Cl₂ was added to the reaction materials which were then shaken vigorously. Thereafter, a CH₂Cl₂ layer was separated and washed with 5% NaHCO₃. An aqueous layer was extracted once again using 30 ml of CH₂Cl₂, washed with 5% NaHCO₃ and combined with the previously extracted organic layer. The organic layer was concentrated and purified by chromatography on silica gel to give 2.31 g of pure Compound 7.

¹H-NMR (CDCl₃, δ): 8.09 (1H, dd, J=1.2, 7.6 Hz), 7.83 (1H, d, J=7.6 Hz), 7.64 (1H, dt, J=1.2, 7.6 Hz), 7.52 (1H, dt, J=1.2, 7.6 Hz), 7.41 (5H, m), 5.27 (1H, dd, J=2.5, 6.0 Hz), 2.77 (1H, m) 2.61 (1H, m), 2.34 (1H, m), 2.08 (1H, m), 0.87 (1H, m)

### Example 8

### Synthesis of Compound 8

The remaining CH₂Cl₂ layer, after extraction with an aqueous 2N NaOH solution in Example 7, was concentrated by distillation under reduced pressure. The resulting concentrates were dissolved in 30 ml of xylene, followed by reflux for 10 hours to induce Claisen Rearrangement. Xylene was concentrated by distillation under reduced pressure and was then mixed with 15 ml of sulfuric acid, without further purification. The resulting mixture was stirred vigorously at room temperature for 10 min and 100 g of ice was added thereto to complete the reaction. 50 ml of CH₂Cl₂ was added to the reaction materials which were shaken vigorously. Thereafter, a CH₂Cl₂ layer was separated and washed with 5% NaHCO₃. An aqueous layer was extracted once again using 20 ml of CH₂Cl₂, washed with 5% NaHCO₃ and combined with the previously extracted organic layer. The organic layer was dried over MgSO₄, concentrated and purified by chromatography on silica gel to give 1.26 g of pure Compound 8.

¹H-NMR (CDCl₃, δ): 8.12 (1H, dd, J=0.8, 8.0 Hz), 7.74 (1H, dd, J=1.2, 7.6 Hz), 7.70 (1H, dt, J=1.2, 7.6 Hz), 7.62 (1H, dt, J=1.6, 7.6 Hz), 7.27 (3H, m), 7.10 (2H, td, J=1.2, 6.4 Hz), 5.38 (1H, qd, J=6.4, 9.2 Hz), 4.61 (1H, d, J=9.2 Hz), 1.17 (3H, d, J=6.4 Hz)

### Example 9

### Synthesis of Compound 9

3.4 g (22 mM) of 1,8-diazabicyclo[5.4.0]undec-7-ene and 1.26 g (15 mM) of 2-methyl-3-butyn-2-ol were dissolved in 10 ml of acetonitrile and the resulting solution was cooled to 0° C. 3.2 g (15 mM) of trifluoroacetic anhydride was gradually added with stirring to the reaction solution which was then continued to be stirred at 0° C. 1.74 g (10 mM) of 2-hydroxy-1,4-naphthoquinone and 135 mg (1.0 mM) of cupric chloride (CuCl₂) were dissolved in 10 ml of acetonitrile in another flask, and were stirred. The previously purified solution was gradually added to the reaction solution which was then refluxed for 20 hours. The reaction solution was concentrated by distillation under reduced pressure and was then purified by chromatography on silica gel to give 0.22 g of pure Compound 9.

¹H-NMR (CDCl₃, 8): 8.11 (1H, dd, J=1.2, 7.6 Hz), 7.73 (1H, dd, J=1.2, 7.6 Hz), 7.69 (1H, dt, J=1.2, 7.6 Hz), 7.60 (1H, dt, J=1.6, 7.6 Hz), 4.95 (1H, d, J=3.2 Hz), 4.52 (1H, d, J=3.2 Hz), 1.56 (6H, s)

### Example 10

### Synthesis of Compound 10

0.12 g of Compound 9 was dissolved in 5 ml of MeOH, 10 mg of 5% Pd/C was added thereto, followed by vigorous stirring at room temperature for 3 hours. The reaction solution was filtered through silica gel to remove 5% Pd/C and was concentrated by distillation under reduced pressure to give Compound 10.

¹H-NMR (CDCl₃, 8): 8.05 (1H, td, J=1.2, 7.6 Hz), 7.64 (2H, m), 7.54 (1H, m), 3.48 (3H, s), 1.64 (3H, s), 1.42 (3H, s), 1.29 (3H, s)

### Example 11

### Synthesis of Compound 11

1.21 g (50 mM) of β-Lapachone (Compound 1) and 1.14 g (50 mM) of DDQ (2,3-dichloro-5,6-dicyano-1,4-benzoqinone) were dissolved in 50 ml of carbon tetrachloride and refluxed for 72 hours. The reaction solution was concentrated by distillation under reduced pressure and was then purified by chromatography on silica gel to give 1.18 g of pure Compound 11.

¹H-NMR (CDCl₃, 8): 8.08 (1H, dd, J=1.2, 7.6 Hz), 7.85 (1H, dd, J=0.8, 7.6 Hz), 7.68 (1H, dt, J=1.2, 7.6 Hz), 7.55 (1H, dt, J=1.2, 7.6 Hz), 6.63 (1H, d, J=10.0 Hz), 5.56 (1H, d, J=10.0 Hz), 1.57 (6H, s)

### Example 12

### Synthesis of Compound 12

1.74 g (10 mM) of 2-hydroxy-1,4-naphthoquinone, 3.4 g (50 mM) of 2-methyl-1,3-butadiene (Isoprene), 3.0 g (100 mM) of paraformaldehyde and 20 ml of 1,4-dioxane were placed into a pressure vessel, and were heated with stirring at 100° C. for 48 hours. The reaction vessel was cooled to mom temperature, and contents therein were filtered. The filtrate was concentrated by distillation under reduced pressure and was then purified by chromatography on silica gel to give 238 mg of Compound 12, as a 2-vinyl derivative of β-Lapachone.

¹H-NMR (CDCl₃, 8): 8.07 (1H, dd, J=1.2, 7.6 Hz), 7.88 (1H, dd, J=0.8, 7.6 Hz), 7.66 (1H, dt, J=1.2, 7.6 Hz), 7.52 (1H, dt, 7.6 Hz), 5.87 (1H, dd, J=10.8, 17.2 Hz), 5.18 (1H, d, J=10.8 Hz), 5.17 (1H, 17.2 Hz), 2.62 (1H, m), 2.38 (1H, m), 2.17 (3H, s), 2.00 (1H, m), 1.84 (1H, m)

### Example 13

### Synthesis of Compound 13

1.74 g (10 mM) of 2-hydroxy-1,4-naphthoquinone, 4.8 g (50 mM) of 2,4-dimethyl-1,3-pentadiene and 3.0 g (100 mM) of paraformaldehyde were dissolved in 20 ml of 1,4-dioxane, and the resulting mixture was refluxed with vigorous stirring for 10 hours. The reaction vessel was cooled to room temperature, and contents therein were filtered to remove paraformaldehyde from solids. The filtrate was concentrated by distillation under reduced pressure and was then purified by chromatography on silica gel to give 428 mg of Compound 13, as a β-Lapachone derivative.

¹H-NMR (CDCl₃, 8): 8.06 (1H, dd, J=1.2, 7.6 Hz), 7.83 (1H, dd, J=0.8, 7.6 Hz), 7.65 (1H, dt, J=1.2, 7.6 Hz), 7.50 (1H, dt, 7.6 Hz), 5.22 (1H, bs), 2.61 (1H, m), 2.48 (1H, m), 2.04 (1H, m), 1.80 (3H, d, J=1.0 Hz), 1.75 (1H, m), 1.72 (1H, d, J=1.0 Hz), 1.64 (3H, s)

### Example 14

### Synthesis of Compound 14

5.3 g (30 mM) of 2-hydroxy-1,4-naphthoquinone, 20.4 g (150 mM) of 2,6-dimethyl-2,4,6-octatriene and 9.0 g (300 mM) of paraformaldehyde were dissolved in 50 ml of 1,4-dioxane, and the resulting mixture was refluxed with vigorous stirring for 10 hours. The reaction vessel was cooled to room temperature, and contents therein were filtered to remove paraformaldehyde from solids. The filtrate was concentrated by distillation under reduced pressure and was then purified by chromatography on silica gel to give 1.18 g of Compound 14, as β-Lapachone derivative.

¹H-NMR (CDCl₃, 8): 8.07 (1H, dd, J=1.2, 7.6 Hz), 7.87 (1H, dd, J=0.8, 7.6 Hz), 7.66 (1H, dt, J=1.2, 7.6 Hz), 7.51 (1H, dt, J=0.8, 7.6 Hz), 6.37 (1H, dd, J=11.2, 15.2 Hz), 5.80 (1H, broad d, J=11.2 Hz), 5.59 (1H, d, J=15.2 Hz), 2.67 (1H, dd, J=4.8, 17.2 Hz), 2.10 (1H, dd, J=6.0, 17.2 Hz), 1.97 (1H, m), 1.75 (3H, bs), 1.64 (3H, bs), 1.63 (3H, s), 1.08 (3H, d, J=6.8 Hz)

### Example 15

### Synthesis of Compound 15

5.3 g (30 mM) of 2-hydroxy-1,4-naphthoquinone, 20.4 g (50 mM) of terpinen and 9.0 g (300 mM) of paraformaldehyde were dissolved in 50 ml of 1,4-dioxane, and the resulting mixture was refluxed with vigorous stiffing for 10 hours. The reaction vessel was cooled to room temperature, and contents therein were filtered to remove paraformaldehyde from solids. The filtrate was concentrated by distillation under reduced pressure and was then purified by chromatography on silica gel to give 1.12 g of Compound 15, as a tetracyclic o-quinone derivative.

¹H-NMR (CDCl₃, δ): 8.06 (1H, d, J=7.6 Hz), 7.85 (1H, d, J=7.6 Hz), 7.65 (1H, t, J=7.6 Hz), 7.51 (1H, t, J=7.6 Hz), 5.48 (1H, broad s), 4.60 (1H, broad s), 2.45 (1H, d, J=16.8 Hz), 2.21 (1H, m), 2.20 (1H, d, J=16.8 Hz), 2.09 (1H, m), 1.77 (1H, m), 1.57 (1H, m), 1.07 (3H, s), 1.03 (3H, d, J=0.8 Hz), 1.01 (3H, d, J=0.8 Hz), 0.96 (1H, m)

### Example 16

### Synthesis of Compounds 16 and 17

17.4 g (0.10M) of 2-hydroxy-1,4-naphthoquinone was dissolved in 120 ml of DMSO, and 0.88 g (0.11M) of LiH was gradually added thereto. Here, this should be done with care because hydrogen evolves. The reaction solution was stirred, and after confirming no further production of hydrogen, was additionally stiffed for another 30 min. Then, 16.3 g (0.12M) of crotyl bromide and 3.35 g (0.025M) of LiI were gradually added thereto. The reaction solution was heated to 45° C. and then vigorously stirred for 12 hours at that temperature. The reaction solution was cooled below 10° C., and 80 g of ice was first added and 250 ml of water was then added. Thereafter, 25 ml of concentrated HCl was gradually added to maintain the resulting solution at an acidic pH>1. 200 ml of CH₂Cl₂ was added to dissolve the reaction mixture which was then shaken vigorously to separate two layers. The aqueous layer was discarded, and a CH₂Cl₂ layer was treated with an aqueous 2N NaOH solution (100 ml×2) to separate the aqueous layer twice. At this time, the remaining CH₂Cl₂ layer after extraction with an aqueous 2N NaOH solution was used in Example 17. The thus-separated aqueous solutions were combined and adjusted to an acidic pH>2 using concentrated HCl, thereby forming solids. The resulting solids were filtered and separated to give a Lapachol derivative. The thus-obtained Lapachol derivative was recrystallized from 75% EtOH. The resulting Lapachol derivative was mixed with 50 ml of sulfuric acid, and the mixture was vigorously stirred at room temperature for 10 min, followed by addition of 150 g of ice to complete the reaction. 60 ml of CH₂Cl₂ was added to the reaction materials which were then shaken vigorously. Thereafter, a CH₂Cl₂ layer was separated and washed with 5% NaHCO₃. An aqueous layer was extracted once again using 30 ml of CH₂Cl₂, washed with 5% NaHCO₃ and combined with the previously extracted organic layer. The organic layer was concentrated and purified by chromatography on silica gel to give 1.78 and 0.43 g of pure Compounds 16 and 17, respectively.

¹H-NMR (CDCl₃, δ) of Compound 16: δ8.07 (1H, dd, J 0.8, 6.8 Hz), 7.64 (2H, broad d, J=3.6 Hz), 7.57 (1H, m), 5.17 (1H, qd, J=6.0, 8.8 Hz), 3.53 (1H, qd, J=6.8, 8.8 Hz), 1.54 (3H, d, 6.8 Hz), 1.23 (3H, d, 6.8 Hz)

¹H-NMR (CDCl₃, δ) of Compound 17: δ8.06 (1H, d, J 0.8, 7.2 Hz), 7.65 (2H, broad d, J=3.6 Hz), 7.57 (1H, m), 4.71 (1H, quintet, J=6.4 Hz), 3.16 (1H, quintet, J=6.4 Hz), 1.54 (3H, d, 6.4 Hz), 1.38 (3H, d, 6.4 Hz)

### Example 17

### Synthesis of Compounds 18 and 19

The remaining CH₂Cl₂ layer, after extraction with an aqueous 2N NaOH solution in Example 16, was concentrated by distillation under reduced pressure. The resulting concentrates were dissolved in 30 ml of xylene, followed by reflux for 10 hours to induce Claisen Rearrangement. Xylene was concentrated by distillation under reduced pressure and was then mixed with 15 ml of sulfuric acid, without further purification. The resulting mixture was stirred vigorously at room temperature for 10 min and 100 g of ice was added thereto to complete the reaction. 50 ml of CH₂Cl₂ was added to the reaction materials which were shaken vigorously. Thereafter, a CH₂Cl₂ layer was separated and washed with 5% NaHCO₃. An aqueous layer was extracted once again using 20 ml of CH₂Cl₂, washed with 5% NaHCO₃ and combined with the previously extracted organic layer. The organic layer was dried over MgSO₄, concentrated and purified by chromatography on silica gel to give 0.62 and 0.43 g of pure Compounds 18 and 19, respectively.

¹H-NMR (CDCl₃, δ) of Compound 18: 8.06 (1H, dd, 7.2 Hz), 7.81 (1H, dd, J=0.8, 7.6 Hz), 7.65 (1H, dt, J=0.8, 7.6 Hz), 7.51 (1H, dt, 7.2 Hz), 4.40 (1H, m), 2.71 (1H, m), 2.46 (1H, m), 2.11 (1H, m), 1.71 (1H, m), 1.54 (3H, d, 6.4 Hz), 1.52 (1H, m)

¹H-NMR (CDCl₃, δ) of Compound 19: 8.08 (1H, d, 7.2 Hz), 7.66 (2H, broad d, J=4.0 Hz), 7.58 (1H, m), 5.08 (1H, m), 3.23 (1H, dd, J=9.6, 15.2 Hz), 2.80 (1H, dd, J=7.2, 15.2 Hz), 1.92 (1H, m), 1.82 (1H, m), 1.09 (3H, t, 7.6 Hz)

### Example 18

### Synthesis of Compound 20

17.4 g (0.10M) of 2-hydroxy-1,4-naphthoquinone was dissolved in 120 ml of DMSO, and 0.88 g (0.11M) of LiH was gradually added thereto. Here, this should be done with care because hydrogen evolves. The reaction solution was stirred, and after confirming no further production of hydrogen, was additionally stirred for another 30 min. Then, 21.8 g (0.10M) of geranyl bromide and 3.35 g (0.025M) of LiI were gradually added thereto. The reaction solution was heated to 45° C. and then vigorously stirred for 12 hours at that temperature. The reaction solution was cooled below 10° C., and 80 g of ice was first added and 250 ml of water was then added. Thereafter, 25 ml of concentrated HCl was gradually added to maintain the resulting solution at an acidic pH>1.200 ml of CH₂Cl₂ was added to dissolve the reaction mixture which was then shaken vigorously to separate two layers. The aqueous layer was discarded, and a CH₂Cl₂ layer was treated with an aqueous 2N NaOH solution (100 ml×2) to separate the aqueous layer twice. The thus-separated aqueous solutions were combined and adjusted to an acidic pH>2 using concentrated HCl, thereby forming solids. The resulting solids were filtered and separated to give 2-geranyl-3-hydroxy-1,4-naphthoquinone. The thus-obtained product was mixed with 50 ml of sulfuric acid without further purification, and the mixture was vigorously stirred at room temperature for 10 min, followed by addition of 150 g of ice to complete the reaction. 60 ml of CH₂Cl₂ was added to the reaction materials which were then shaken vigorously. Thereafter, a CH₂Cl₂ layer was separated and washed with 5% NaHCO₃. An aqueous layer was extracted once again using 30 ml of CH₂Cl₂, washed with 5% NaHCO₃ and combined with the previously extracted organic layer. The organic layer was concentrated and purified by chromatography on silica gel to give 3.62 g of pure Compound 20.

¹H-NMR (CDCl₃, δ): 8.05 (1H, d, J=7.6 Hz), 7.77 (1H, d, J=7.6 Hz), 7.63 (1H, t, J=7.6 Hz), 7.49 (1H, t, J=7.6 Hz), 2.71 (1H, dd, J=6.0, 17.2 Hz), 2.19 (1H, dd, J=12.8, 17.2 Hz), 2.13 (1H, m), 1.73 (2H, m), 1.63 (1H, dd, J=6.0, 12.8 Hz), 1.59 (1H, m), 1.57 (1H, m), 1.52 (1H, m), 1.33 (3H, s), 1.04 (3H, s), 0.93 (3H, s)

### Example 19

### Synthesis of Compound 21

Compound 21 was obtained in the same manner as in Example 1, except that 6-chloro-2-hydroxy-1,4-naphthoquinone was used instead of 2-hydroxy-1,4-naphthoquinone.

¹H-NMR (CDCl₃, δ): 8.02 (1H, d, J=8 Hz), 7.77 (1H, d, J=2 Hz), 7.50 (1H, dd, J=2, 8 Hz), 2.60 (2H, t, J=7 Hz), 1.87 (2H, t, J=7 Hz) 1.53 (6H, s)

### Example 20

### Synthesis of Compound 22

Compound 22 was obtained in the same manner as in Example 1, except that 2-hydroxy-6-methyl-1,4-naphthoquinone was used instead of 2-hydroxy-1,4-naphthoquinone.

¹H-NMR (CDCl₃, δ): 7.98 (1H, d, J=8 Hz), 7.61 (1H, d, J=2 Hz), 7.31 (1H, dd, J=2, 8 Hz), 2.58 (2H, t, J=7 Hz), 1.84 (2H, t, J=7 Hz) 1.48 (6H, s)

### Example 21

### Synthesis of Compound 23

Compound 23 was obtained in the same manner as in Example 1, except that 6,7-dimethoxy-2-hydroxy-1,4-naphthoquinone was used instead of 2-hydroxy-1,4-naphthoquinone.

¹H-NMR (CDCl₃, 8): 7.56 (1H, s), 7.25 (1H, s), 3.98 (6H, s), 2.53 (2H, t, J=7 Hz), 1.83 (2H, t, J=7 Hz) 1.48 (6H, s)

### Example 22

### Synthesis of Compound 24

Compound 24 was obtained in the same manner as in Example 1, except that 1-bromo-3-methyl-2-pentene was used instead of 1-bromo-3-methyl-2-butene.

¹H-NMR (CDCl₃, 8): 7.30-8.15 (4H, m), 2.55 (2H, t, J=7 Hz), 1.83 (21-1, t, J=7 Hz), 1.80 (2H, q, 7 Hz) 1.40 (3H, s), 1.03 (3H, t, J=7 Hz)

### Example 23

### Synthesis of Compound 25

Compound 25 was obtained in the same manner as in Example 1, except that 1-bromo-3-ethyl-2-pentene was used instead of 1-bromo-3-methyl-2-butene.

¹H-NMR (CDCl₃, δ): 7.30-8.15 (4H, m), 2.53 (2H, t, J=7 Hz), 1.83 (2H, t, J=7 Hz), 1.80 (4H, q, 7 Hz) 0.97(6H, t, J=7 Hz)

### Example 24

### Synthesis of Compound 26

Compound 26 was obtained in the same manner as in Example 1, except that 1-bromo-3-phenyl-2 butene was used instead of 1-bromo-3-methyl-2-butene.

¹H-NMR (CDCl₃, 8): 7.15-8.15 (9H, m), 1.90-2.75 (4H, m), 1.77 (3H, s)

### Example 25

### Synthesis of Compound 27

Compound 27 was obtained in the same manner as in Example 1, except that 2-bromo-ethylidenecyclohexane was used instead of 1-bromo-3-methyl-2-butene.

¹H-NMR (CDCl₃, δ): 7.30-8.25 (4H, m), 2.59 (2H, t, J=7 Hz), 1.35-2.15 (12H, m)

### Example 26

### Synthesis of Compound 28

Compound 28 was obtained in the same manner as in Example 1, except that 2-bromo-ethylidenecyclopentane was used instead of 1-bromo-3-methyl-2-butene.

¹H-NMR (CDCl₃, δ): 7.28-8.20 (4H, m), 2.59 (2H, t, J=7 Hz), 1.40-2.20 (10H, m)

### Example 27

### Synthesis of Compound 29

8.58 g (20 mM) of Compound 5 synthesized in Example 5 was dissolved in 1000 ml of carbon tetrachloride, followed by addition of 11.4 g (50 mM) of 2,3-dichloro-5,6-dicyano-1,4-benzoqinone, and the resulting mixture was refluxed for 96 hours. The reaction solution was concentrated by distillation under reduced pressure and the resulting red solids were then recrystallized from isopropanol, thereby obtaining 7.18 g of pure Compound 29.

¹H-NMR (CDCl₃, 8): 8.05 (1H, dd, J=1.2, 7.6 Hz), 7.66 (1H, dd, J=1.2, 7.6 Hz), 7.62 (1H, dt, J=1.2, 7.6 Hz), 7.42 (1H, dt, J=1.2, 7.6 Hz), 6.45 (1H, q, J=1.2 Hz), 2.43 (3H, d, J=1.2 Hz)

### Example 28

### Synthesis of Compound 30

Analogous to a synthesis method as taught in J. Org. Chem., 55 (1990) 4995-5008,4,5-dihydro-3-methylbenzo[1,2-b]furan-4,5-dione {Benzofuran-4,5-dione} was synthesized using p-benzoquinone and 1-(N-morpholine)propene. 1.5 g (9.3 mM) of the thus-prepared benzofuran-4,5-dione and 3.15 g (28.2 mM) of 1-acetoxy-1,3-butadiene were dissolved in 200 ml of benzene, and the resulting mixture was refluxed for 12 hours. The reaction solution was cooled to mom temperature and concentrated by distillation under reduced pressure. This was followed by chromatography on silica gel to give 1.13 g of pure Compound 30.

¹H-NMR (CDCl₃, 5): 8.05 (1H, dd, J=1.2, 7.6 Hz), 7.68 (1H, dd, J=1.2, 7.6 Hz), 7.64 (1H, td, J=1.2, 7.6 Hz), 7.43 (1H, td, J=1.2, 7.6 Hz), 7.26 (1H, q, J=1.2 Hz), 2.28 (3H, d, J=1.2 Hz)

### Example 29

### Synthesis of Compounds 31 and 32

1.5 g (9.3 mM) of 4,5-dihydro-3-methylbenzo[1,2-b]furan-4,5-dione {Benzofuran-4,5-dione} and 45 g (0.6M) of 2-methyl-1,3-butadiene were dissolved in 200 ml of benzene, and the resulting mixture was refluxed for 5 hours. The reaction solution was cooled to room temperature and completely concentrated by distillation under reduced pressure. The thus-obtained concentrates were dissolved again in 150 ml of carbon tetrachloride, followed by addition of 2.3 g (10 mM) of 2,3-dichloro-5,6-dicyano-1,4-benzoqinone, and the resulting mixture was further refluxed for 15 hours. The reaction solution was cooled and concentrated by distillation under reduced pressure. The resulting concentrates were purified by chromatography on silica gel to give 0.13 g and 0.11 g of pure Compounds 31 and 32, respectively.

¹H-NMR (CDCl₃, δ) of Compound 31: 7.86 (1H, s), 7.57 (1H, d, J=8.1 Hz), 7.42 (1H, d, J=8.1 Hz), 7.21 (1H, q, J=1.2 Hz), 2.40 (3H, s), 2.28 (1H, d, J=1.2 Hz) (CDCl₃, δ) of Compound 32: δ7.96 (1H, d, J=8.0 Hz), 7.48 (1H, s), 7.23 (2H, m), 2.46 (3H, s), 2.28 (1H, d, J=1.2 Hz)

Hereinafter, objects and methods used in the present invention will be given as follows.

### 1. Cell Culture

For vascular smooth muscle cell cultivation, vascular smooth muscle cells were isolated from rat aorta and they were primarily cultured. The vascular smooth muscle cells were cultured and grown in a culture solution containing 20% fetal bovine serum (FBS) at 37° C. under 5% CO₂. The cells obtained in this process were transferred to a new culture plate for experiments. The cells used in the experiments were initial cells which had been subcultured 4 to 7 times. In order to activate NQO1, the vascular smooth muscle cells, when the cell mass reached 80 to 90%, were cultured in a medium containing 0.5% fetal bovine serum for 24 hours to keep the cells in the resting state. Such cells were treated with the compound of Example 1 (hereinafter, referred to as Compound 1).

### 2. Examination of Proliferation Rate of Vascular Smooth Muscle Cells

Primary cultures of vascular smooth muscle cells were seeded onto 96-well plates, cultured to reach cell mass of 70%, and transferred and again cultured in a medium containing 0.5% fetal bovine serum for 24 hours. Then, the cells were kept in the resting state. Thereafter, the cells were treated with platelet derived growth factor (PDGF) and Compound 1, and reacted at 37° C. for 48 hours. Hereto, a reagent for confirming cell proliferation was treated. After further reacting for 4 hours, absorbencies were measured at 450 nm using an ELISA reader to examine the cell proliferation rate.

### 3. RT-PCR

Primary cultures of vascular smooth muscle cells were treated with Compound 1 at 37° C. and reacted for a predetermined period of time. RNA was extracted using trizol, and then reversely transcribed into cDNA. Using NQO1 primer, the constructed cDNA was amplified and then subjected to electrophoresis to confirm the expression of NQO1.

### 4. Western Blot

The vascular smooth muscle cells collected after reacting with Compound 1 were lysed in RIPA buffer (50 mM Tris-HCl, 150 mM NaCl, 5 mM EDTA, 1% NP-40, 1 mM PMSF, 1 mM MT, and 1 mg/ml protease inhibitor) to isolate whole proteins. The isolated proteins of each sample were quantified. 25 μg of the proteins were mixed with a sample buffer and boiled for 5 minutes. The boiled proteins were cooled and subjected to electrophoresis on a sodium dodecyl sulfate polyacrylamide gel, thereby separating the proteins by their sizes. These proteins were again transferred to PVDF membrane and immunoreacted with antibodies against pAMPK, pACC, p53, p21, CDK and pRb to confirm the protein expression. In addition, in order to verify whether an equal amount of protein was used, the proteins were reacted with anti-β-actin.

### 5. Cell Cycle Analysis

FACS was used for cell cycle analysis. The vascular smooth muscle cells incubated in a medium containing 0.5% fetal bovine serum for 24 hours were pretreated with Compound 1 for 2 hours. The cells were treated with PDGF and insulin to induce cell proliferation and reacted for 48 hours. Thereafter, the cells were collected and passed through fixation process, followed by nuclear staining with propidium iodide (PI). Then, the cell cycle was analyzed using FACS.

### 6. Animal Experimentation

SD rats were used for performing balloon angioplasty. Animals were housed in a breeding room maintained at a constant temperature of 22±2° C. and a 12-h light/dark (UD) cycle. The animals were divided into two groups, i.e., a control group with administration of general diet and an experiment group with administration of 100 mg/kg of Compound 1, having 4 animals in each group. The 4 animals in each group were raised in separate cages for each animal for 4 weeks, during which experiments were carried out. The animals were raised for 2 weeks before performing balloon angioplasty and 2 weeks after performing balloon angioplasty while continuing the dietary intake of the general diet and Compound 1 diet. Thereafter, their aortas were isolated to confirm hyoerplasia by means of H&E (hematoxylin and eosin) staining.

### 7. Measurement of Lipogenesis

The animals were killed to isolate their hearts and abdominal aortas, and they were fixed in 4% formalin, respectively. The fixed heart tissue was immersed in an isotonic sucrose solution, and then stored in a freezer by embedding in an OCT compound. Meanwhile, the fixed abdominal aorta was incised lengthwise and prepared such that the internal vessel was exposed. The frozen heart tissue was sectioned into a thickness of 20 μm and attached onto a coating slide, while the abdominal aorta was immersed in an Oil Red O solution to perform Oil Red O staining, and they were observed.

### Experimental Example 1

### Effects of Compound 1 on Vascular Smooth Muscle Cell Proliferation

In order to determine the effects of Compound 1 on vascular smooth muscle cell proliferation, the cells cultured in a 96-well plate were kept in the resting state, followed by pre-treating them with Compound 1 by varying the concentration, and by reacting them with PDGF for 48 hours. The number of living cells was counted using a WST cell counting kit. The number was counted in 3 or more independent experiments to calculate the mean number.

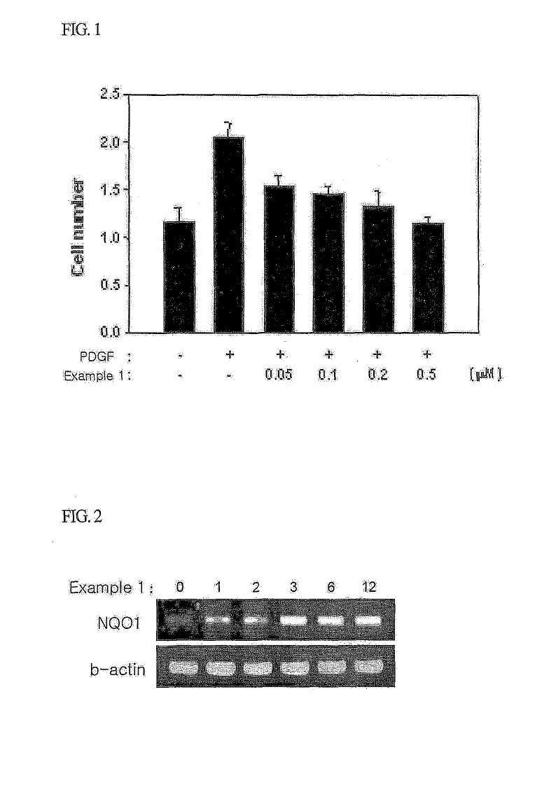

The results of the measurement on living cells are presented in FIG. 1. Referring to FIG. 1, the number of the vascular smooth muscle cells treated with PDGF and Compound 1 was significantly low compared with that treated only with PDGF. This number was similar to the cell number of the control group without the treatment of PDGF and Compound 1 (PDGF: -, Compound 1: -). In addition, it was known that a degree of reduction of the number of vascular smooth muscle cells increased as the administration dose of Compound 1 increased. As a result, it is believed that the reduction in the number of vascular smooth muscle cells is concentration-dependent according to the Compound 1 administration.

Therefore, Compound 1 shows efficacy of reducing the number of vascular smooth muscle cells. Thus, Compound 1 is believed to have excellent effects on the treatment of restenosis or arteriosclerosis associated with fast increase in the number of vascular smooth muscle cells.

### Experimental Example 2

### Effects of Compound 1 on Expression of NQO1

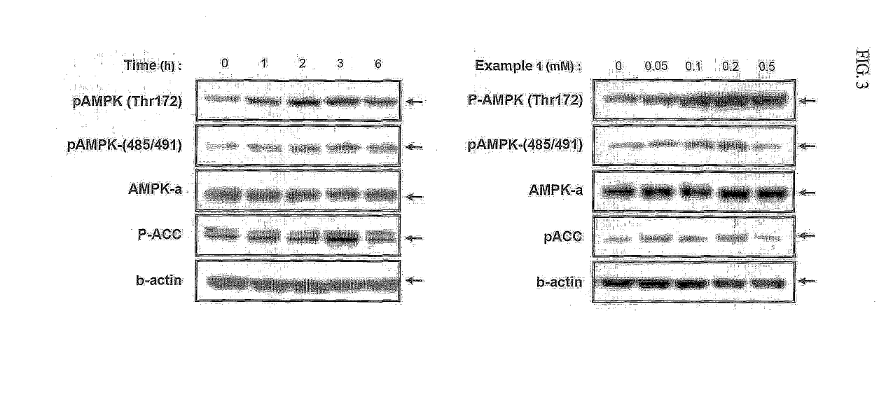

A study was conducted to investigate effects of Compound 1 on the expression of NQ01 as in the following. Primary cultures of vascular smooth muscle cells were kept in the resting state and then treated with 0.5 μM of Compound 1. After reacting them for a predetermined period of time, RNA was extracted from the cells using trizol. Thereafter, DNA was amplified using NQO1 primer and then subjected to electrophoresis to confirm the expression level of NQO1. The results are presented in FIG. 2. Referring to FIG. 2, it was confirmed that the expression level of NQO1 in the vascular smooth muscle cells was significantly higher than that compared with the control group. As a result, Compound 1 is believed to have a function as an NQO 1 activator.

### Experimental Example 3

### Effects of Compound 1 on Phosphorylation of AMPK and ACC

This Example was intended to investigate a mechanism of Compound 1 functioning as a therapeutic agent for restenosis, and the experiment is carried out as in the following.

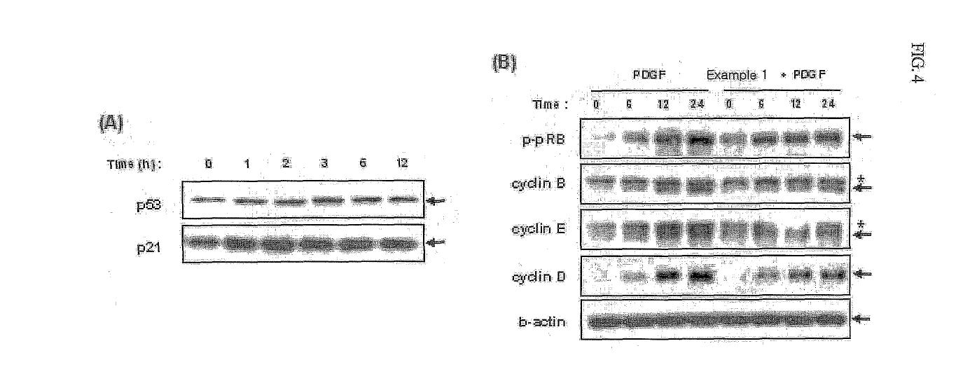

Primary cultures of vascular smooth muscle cells were kept in the resting state and then treated with 0.5 μM of Compound 1. After reacting them for a predetermined period of time, the cells were collected to perform western blot. Quantification was carried out using antibodies specific for phosphorylated AMPK and Acetyl-CoA carboxylase (ACC), and for β-actin. The results were confirmed through three or more repeating experiments. The results are presented in FIG. 3.

Referring to FIG. 3, a degree of AMPK and ACC phosphorylation by Compound 1, when compared with the control group, exhibited increased AMPK phosphorylation, whereas exhibited decreased ACC phosphorylation. The phosphorylated AMPK could be observed 1 hour after the treatment with Compound 1, and last for 6 hours. This is because, Compound 1, reducing the number of vascular smooth muscle cell, elevated NAD⁺ in the cells by activating NQO1, which then also activated AMPK. Moreover, it was confirmed that the reason for the decrease in a phosphorylation degree of ACC, which is known to be a target protein of AMPK, is because Compound 1 activated AMPK, thereby inhibiting activity of ACC, which is an important regulation enzyme for lipogenesis, and increasing lipid metabolism.

Therefore, it can be seen that the mechanism of Compound 1 functioning as a therapeutic agent of restenosis and arteriosclerosis is such that Compound 1 increases activities of NQO1 and AMPK resulting in inhibition of vascular smooth muscle cell proliferation and decreases ACC activity resulting in inhibition of lipogenic activity.

### Experimental Example 4

### Effects of Compound 1 on Changes in Expressions of p53 and p21, and Changes in Expressions of RB and CDK

Primary cultures of vascular smooth muscle cells were treated with 0.5 μM of Compound 1. After reacting them for a predetermined period of time, the cells were collected to perform western blot. Expressions of p53 and p21 (FIG. 4A) and expressions of phosphorylated retinoblastoma (RB) and cyclin dependent kinase (CDK) (FIG. 4B) were also confirmed with 25 mg of cell debris. The results were confirmed through three or more repeating experiments. The results are presented in FIG. 4.

By measuring the expression levels of the cell cycle regulating proteins, such as CDK, retinoblastoma protein, p53, and downstream p21 controlled according to the expression level of p53, whether cell proliferation has progressed can be confirmed. This has close relation with inhibiting the vascular smooth muscle cell proliferation.

Referring to FIG. 4A, when treating the proteins with Compound 1, the expression level of p53 did not increase largely, but it was confirmed that p21 increased in a great amount correlated with the expression of p53. This indicates that the cell cycle can be inhibited by inducing the expression of p21, which is a downstream protein of p53.

Referring to FIG. 4B, when treating the proteins with Compound 1, CDK is activated through phosphorylation. Thus, it can be seen that the expression levels of cyclin D, cyclin E, and cyclin B are all significantly low compared with those of the control group. Here, the cyclin D is known to serve an important role in initiating the cell cycle, cyclin E is known as an essential factor for G1-S transition by being expressed at the last stage of G1 phase to bond with CDK2, and cyclin B accelerates the progression of cell cycle to M phase.

Therefore, when treating the vascular smooth muscle cells with Compound 1, it can be seen that cell proliferation is inhibited through regulating the progression of the cell cycle. This indicates that Compound 1 is believed to have effects on inhibition of vascular smooth muscle cell proliferation. Thus, it can be confirmed that Compound 1 can be effectively used for treatment of restenosis associated with vascular smooth muscle cell proliferation.

### Experimental Example 5

### Effects of Compound 1 on Cell Cycle

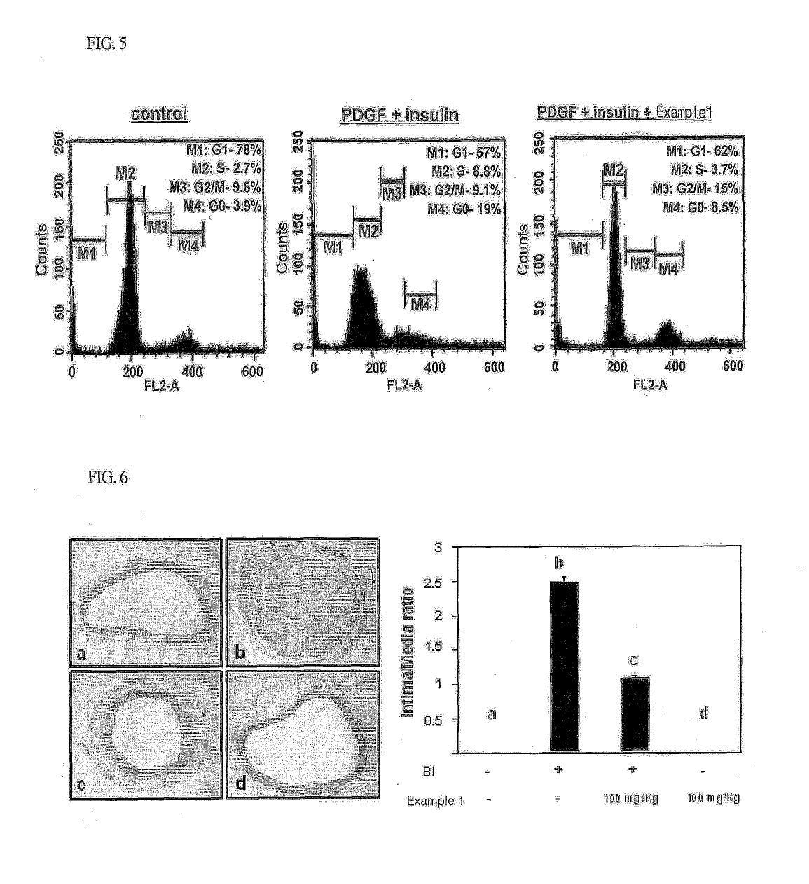

Primary cultures of vascular smooth muscle cells were treated with 0.5 μM of Compound 1, followed by reacting with PDGF and insulin for 48 hours. After reacting for a predetermined period of time, the cells were fixed and stained with propidium iodide (PI). 10,000 cells were counted for each sample, and each phase in the cell cycle is presented in percentages, as presented in FIG. 5.

Referring to FIG. 5, G1 phase of FIG. 5C with treatment of Compound 1, PDGF and insulin is 62%, whereas G1 phase of FIG. 5B with treatment of only PDGF and insulin is 57%. This shows that G1 phase of FIG. 5C is further increased compared with G1 phase of FIG. 5B.

G1 phase is a stage for determining whether cell proliferation has been inhibited or not Thus, increase in G1 phase implies that vascular smooth muscle cell proliferation is inhibited by the treatment with Compound 1 since G1 phase is not progressed to S phase. Therefore, it is confirmed that Compound 1 is effective for inhibition of vascular smooth muscle cell proliferation. As a result, Compound 1 may be used as a composition for treatment of arteriosclerosis and restenosis associated with vascular smooth muscle cell proliferation.

### Experimental Example 6

### Effects of Compound 1 on Intimal Hyperplasia

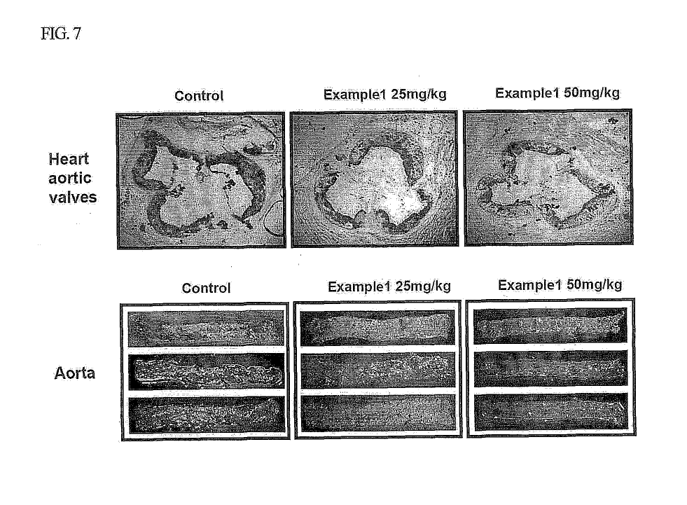

14 days after performing balloon angioplasty, the carotid artery was extracted from a rat. After staining the carotid artery by H&E staining, vessel intimal hyperplasia was observed. The results are presented in FIG. 6. a is a control group, b is a vessel intima after performing balloon angioplasty in the general dietary group, c is a vessel intima after performing balloon angioplasty in the 100 mg/kg of Compound 1 administration group, and d is a control group in the 100 mg/kg of Compound 1 administration group, without performing balloon angioplasty.

As seen from b and c of FIG. 6, the general dietary group b shows restenosis due to intimal hyperplasia in the vessel after performing balloon angioplasty. On the other hand, the Compound 1 administration group c shows sufficiently ensured blood flow way due to a low rate of intimal hyperplasia. In addition, when examining a intima/media ratio, it can be seen that the intima/media ratio of the general dietary group b is 2.5, whereas the Compound 1 administration group c is 1, which is a far less value compared with the group b. This implies that the group c has significantly low rate of intimal hyperplasia. Therefore, it is believed that Compound 1 can be a therapeutically effective agent for resolving problems of restenosis induced by the intimal hyperplasia after performing a surgical operation on arteriosclerosis or the like.

### Experimental Example 7

### Effects of Compound 1 on Lipogenesis in Vessels

Rats administered with 25 mg/kg and 50 mg/kg of Compound 1, respectively, for 4 weeks had been killed, and their hearts and abdominal aortas were extracted to prepare frozen sections. The prepared sections were stained with Oil Red O, and a degree of lipid accumulation on the inner vessel wall and aortic valve was compared/observed.

A (upper part of FIG. 7) is an aortic valve and B (bottom part of FIG. 7) is an abdominal aorta. The experiments were repeated three times to confirm the results. The results are presented in FIG. 7.

Referring to FIG. 7, it was observed that when treated with Compound 1, lipogenesis in both A and B were inhibited and the blood flow began to increase. It was also observed that the blood flow level in the heart and abdominal aorta of the rats with administration of about 50 mg/kg of Compound 1 exhibited approximately the same as that of the normal rats. Therefore, Compound 1 is believed to have effects on inhibition of lipogenesis in blood vessels and can be used effectively for substantial treatment of arteriosclerosis associated with rapid proliferation of the vascular smooth muscle cells due to vessel wall injury by lipids.

As apparent from the above description, the pharmaceutical composition of the present invention can function effectively on inhibition of rapid proliferation of vascular smooth muscle cells. Therefore, the composition has an excellent effect on substantial treatment and/or prevention of restenosis generated after surgical operation associated with vascular smooth muscle cell proliferation.

### INDUSTRIAL APPLICABILITY

As apparent from the above description, a pharmaceutical composition according to the present invention is effective in inhibition of vascular smooth muscle cell proliferation. Accordingly, a pharmaceutical composition according to the present invention is effective in fundamental prevention and treatment of restenosis generated after surgical operation associated with vascular smooth muscle cell proliferation.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A pharmaceutical composition for the treatment and/or prevention of restenosis, comprising:
(a) a therapeutically effective amount of one or more compounds selected from the compounds represented by Formula 1 and Formula 2 below, or a pharmaceutically acceptable salt, prodrug, solvate or isomer thereof: 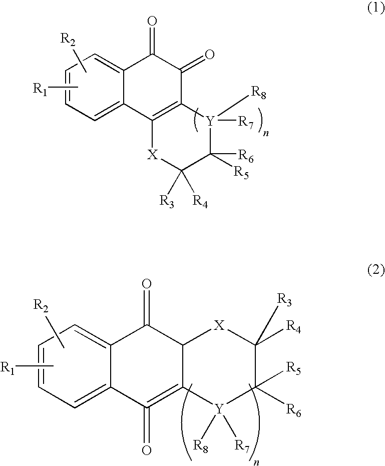
wherein
R₁ and R₂ are each independently hydrogen, halogen, amino, alkoxy, or C₁-C₆ lower alkyl or alkoxy, or R₁ and R₂ may be taken together to form a substituted or unsubstituted cyclic structure which may be saturated or partially or completely unsaturated;
R₃, R₄, R₅, R₆, R₇ and R₈ are each independently hydrogen, hydroxyl, amino, C₁-C₂₀ alkyl, alkene or alkoxy, C₄-C₂₀ cycloalkyl, heterocycloalkyl, aryl or heteroaryl, or two substituents of R₃ to R₈ may be taken together to form a cyclic structure which may be saturated or partially or completely unsaturated;
X is selected from a group consisting of C(R)(R′), N(R″), O and S, preferably O or S, and more preferably O, wherein R′ is hydrogen or C₁-C₆ lower alkyl;
Y is C, S or N, with proviso that when Y is S, R₇ and R₁ are nothing and when Y is N, R₇ is hydrogen or C₁-C₆ lower alkyl and R₈ is nothing; and
n is 0 or 1, with proviso that when n is 0, carbon atoms adjacent to n form a cyclic structure via a direct bond; and
(b) a pharmaceutically acceptable carrier, a diluent or an excipient, or any combination thereof.

2. The composition according to claim 1, wherein X is O.

3. The composition according to claim 1, wherein the prodrug is a compound represented by Formula 1a below: 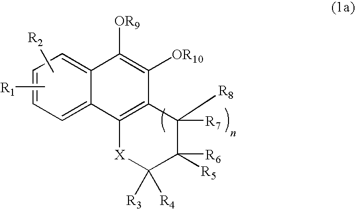
wherein,
R₁, R₂, R₃, R₄, R₅, R₆, R₇, R₈, X and n are as defined in Formula 1;
R₉ and R₁₀ are each independently —SO₃⁻Na⁺ or substituent represented by Formula A below or a salt thereof, 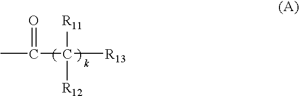
wherein,
R₁₁ and R₁₂ are each independently hydrogen, or substituted or unsubstituted C₁-C₂₀ linear alkyl or C₁-C₂₀ branched alkyl
R₁₃ is selected from the group consisting of substituents i) to viii) below:
i) hydrogen;
ii) substituted or unsubstituted C₁-C₂₀ linear alkyl or C₁-C₂₀ branched alkyl;
iii) substituted or unsubstituted amine;
iv) substituted or unsubstituted C₃-C₁₀ cycloalkyl or C₃-C₁₀ heterocycloalkyl;
v) substituted or unsubstituted C₄-C₁₀ aryl or C₄-C₁₀ heteroaryl;
vi) —(CRR′—NR″CO)ₗ—R₁₄, wherein, R, R′ and R″ are each independently hydrogen, or substituted or unsubstituted C₁-C₂₀ linear alkyl or C₁-C₂₀ branched alkyl, R₁₄ is selected from the group consisting of hydrogen, substituted or unsubstituted amine, cycloalkyl, heterocycloalkyl, aryl and heteroaryl, and 1 is selected from the 1˜5;
vii) substituted or unsubstituted carboxyl;
viii) —OSO₃⁻Na⁺;
k is selected from the 0˜20, with proviso that when k is 0, R₁₁ and R₁₂ are not anything, and R₁₃ is directly bond to a carbonyl group.

4. The composition according to claim 1, wherein the compound of Formula 1 is selected from compounds of Formulas 3 and 4 below: 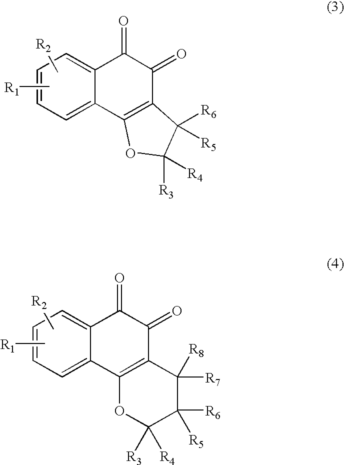
wherein, R₁, R₂, R₃, R₄, R₅, R₆, R₇ and R₈ are defined as in the Formula 1.

5. The composition according to claim 1, wherein each of R₁ and R₂ is hydrogen.

6. The composition according to claim 4, wherein the compound of Formula 3 is the compound of Formula 3a below in which R₁, R₂ and R₄ are respectively hydrogen, or the compound of Formula 3b below in which R₁, R₂ and R₆ are respectively hydrogen:

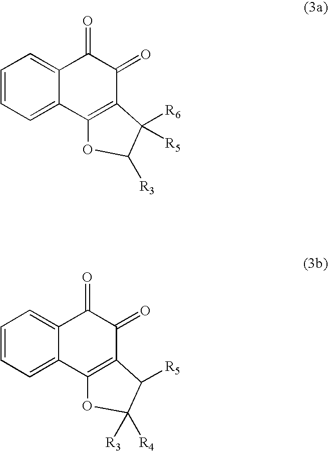

7. The composition according to claim 4, wherein the compound of Formula 4 is the compound selected from compounds of Formulas 4a, 4b and 4c below:

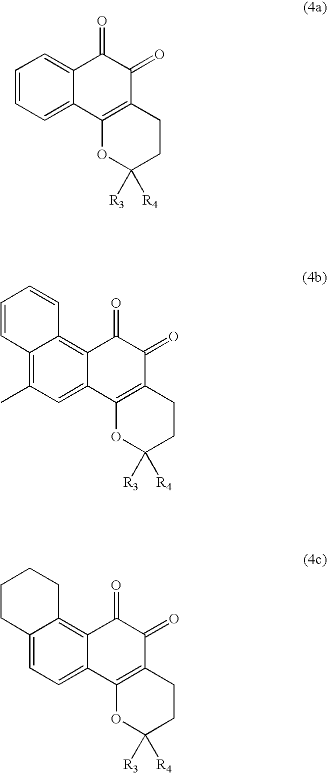

8. The composition according to claim 1, wherein the compound of Formula 2 is the compound of Formula 2a wherein n is 0 and adjacent carbon atoms form a cyclic structure via a direct bond therebetween and Y is C, or the compound of Formula 2b wherein n is 1 and Y is C. 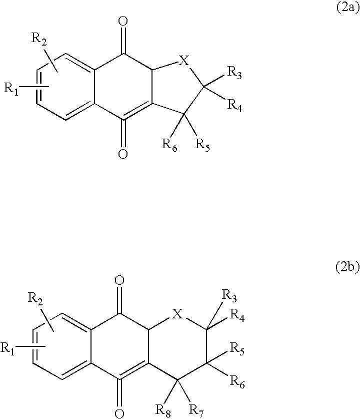
wherein, R₁, R₂, R₃, R₄, R₅, R₆, R₇, R₈ and X are defined as in the Formula 1.

9. The composition according to claim 1, wherein the compound of Formula 1 or 2 has a crystalline structure.

10. The composition according to claim 1, wherein the compound of Formula 1 or 2 has an amorphous structure.

11. The composition according to claim 1, wherein the compound of Formula 1 or 2 is formulated into the form of a fine particle.

12. The composition according to claim 11, wherein the formulation for form of a fine particle is carried out by the particle micronization method selected from the group consisting of mechanical milling, spray drying, precipitation method, high-pressure homogenization, and supercritical micronization.

13. The composition according to claim 12, wherein the formulation is carried out by jet milling as a mechanical milling and/or spray drying.

14. The composition according to claim 11, the particle size of fine particles is within a range of 5 nm to 500 μm.

15. The composition according to claim 1, wherein the composition is prepared into an intestine-targeted formulation.

16. The composition according to claim 15, wherein the intestine-targeted formulation is carried out by addition of a pH sensitive polymer.

17. The composition according to claim 15, wherein the intestine-targeted formulation is carried out by addition of a biodegradable polymer which is decomposable by an intestine-specific bacterial enzyme.

18. The composition according to claim 15, wherein the intestine-targeted formulation is carried out by addition of a biodegradable matrix which is decomposable by an intestine-specific bacterial enzyme.

19. The composition according to claim 15, wherein the intestine-targeted formulation is carried out by a configuration with time-course release of the drug after a lag time (‘time-specific delayed-release formulation’).

20. The composition according to claim 1, wherein the compound of Formula 1 or Formula 2 is added by being coated on or embedded in a mesh stent to be inserted in blood vessels.

21. A use of a compound represented by Formula 1 or Formula 2 according to claim 1 in the preparation of a drug for preventing and treating diseases associated with rapid proliferation of vascular smooth muscle cell.

22. The use according to claim 21, wherein the disease is restenosis.
